(19) United States
(12) Patent Application Publication
(10) Pub. No.: US 2026/0364601 A1
(43) Pub. Date: Nov. 19, 2026

(54) SYSTEM FOR DOSE FRACTIONATION OF RADIOACTIVE MICROSPHERES WITH MICROFLUIDIC CASSETTE

(71) Applicant: Boston Scientific Scimed, Inc., Maple Grove, MN (US)

(72) Inventors: Kyle True, Minneapolis, MN (US); Zachary Nickle, Minneapolis, MN (US); Mark Steven Smith, Coon Rapids, MN (US); Newell G. Voss, St. Paul, MN (US); Ramon Libarnes, Dayton, MN (US)

(21) Appl. No.: 19/677,563

(22) Filed: May 14, 2026

Related U.S. Application Data
(60) Provisional application No. 63/807,329, filed on May 16, 2025.

(51) Int. Cl. B01L 3/00

(52) U.S. Cl. CPC B01L 3/502761; B01L 3/502715; B01L 2200/0647; B01L 2300/049; B01L 2300/0816; B01L 2400/086

(57) ABSTRACT

Microfluidic fractionation systems are provided herein for cancer therapy using radioactive microspheres. The system comprises a microfluidic cassette including a microsphere conveying channel, a microsphere dam configured to block microspheres and allow carrier fluid passage, and a weep channel separated from the conveying channel by the dam. The cassette further includes a fractionation output valve and a weep channel clearance valve, under the control of a circuit programmed to coordinate their operation. In some embodiments, the system comprises a holding tank and a mixing chamber configured for pressure (positive, negative, or both) driven transfer of a microsphere suspension. The system facilitates accumulation of a selected fraction of radioactive microspheres in the conveying channel, removal of carrier fluid, and controlled transfer to a holding tank or delivery catheter. Variations include multi-layer cassette construction, integration of pressure or flow sensors, and automated valve actuation for sequential or multi-site dosing during therapeutic procedures.

This application claims the benefit of U.S. Provisional Application No. 63/807,329, filed May 16, 2025, the content of which is herein incorporated by reference in its entirety.

### FIELD

Embodiments herein relate to systems for dose fractionation of radioactive microspheres for cancer therapy. In particular, embodiments herein relate to systems for dose fractionation of radioactive microspheres including a microfluidics cassette.

### BACKGROUND

Radioactive microspheres have been developed as a means of treating cancer by delivering targeted radiation therapy directly to tumors. These microspheres, which contain therapeutic radioisotopes, can be administered using systems that infuse them through a delivery catheter for precise delivery to the target treatment site. For example, Yttrium-90 (Y-90) glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy. This targeted therapeutic approach can maximize the radiation dose at the tumor site while minimizing exposure to healthy surrounding tissue. Despite ongoing advancements, challenges remain in improving the precision and adaptability of these systems across diverse clinical applications.

### SUMMARY

Embodiments herein relate to systems for dose fractionation of radioactive microspheres including a microfluidics cassette. In a first aspect, a fractionation system for microspheres for cancer therapy can be included having a microfluidic cassette. The microfluidic cassette can include a microsphere conveying channel and a microsphere dam. The microsphere dam can have a physical geometry configured to block microspheres from passing through the microsphere dam and a weep channel, wherein the weep channel can be separated from the microsphere conveying channel by the microsphere dam. A fractionation output valve can be included wherein the fractionation output valve can be in fluid communication with the microsphere conveying channel. A weep channel clearance valve can be included, wherein the weep channel clearance valve is in fluid communication with the weep channel. In addition, a control circuit can be included that is configured to control the fractionation output valve and the weep channel clearance valve.

In a second aspect, in addition to one or more of the preceding or following aspects, or in the alternative to some aspects, the fractionation output valve can be a pneumatically operated valve.

In a third aspect, in addition to one or more of the preceding or following aspects, or in the alternative to some aspects, the weep channel clearance valve can be a pneumatically operated valve.

In a fourth aspect, in addition to one or more of the preceding or following aspects, or in the alternative to some aspects, the microsphere dam can have a height, width, and length dimension configured to allow fluid to pass into the weep channel while blocking passage of microspheres.

In a fifth aspect, in addition to one or more of the preceding or following aspects, or in the alternative to some aspects, the microsphere dam can have a cross-sectional profile with a minimum aperture dimension size less than 20 micrometers.

In a sixth aspect, in addition to one or more of the preceding or following aspects, or in the alternative to some aspects, the system can further include a holding tank, wherein the holding tank can be in fluid communication with the microsphere conveying channel as interrupted by the fractionation output valve.

In a seventh aspect, in addition to one or more of the preceding or following aspects, or in the alternative to some aspects, the fractionation system for microspheres for cancer therapy can further include a catheter connection port, and a holding tank output valve, wherein the holding tank can be in fluid communication with the catheter connection port as interrupted by the holding tank output valve.

In an eighth aspect, in addition to one or more of the preceding or following aspects, or in the alternative to some aspects, can further include a plurality of catheter connection ports.

In a ninth aspect, in addition to one or more of the preceding or following aspects, or in the alternative to some aspects, the system can further include a mixing chamber, wherein the mixing chamber can be configured to supply a suspension of therapeutic radioactive microspheres to the microfluidic cassette.

In a tenth aspect, in addition to one or more of the preceding or following aspects, or in the alternative to some aspects, the fractionation system for microspheres for cancer therapy can be operable in a positive pressure mode in which a positive pressure can be applied to move the suspension of therapeutic radioactive microspheres from the mixing chamber to the microfluidic cassette.

In an eleventh aspect, in addition to one or more of the preceding or following aspects, or in the alternative to some aspects, the fractionation system for microspheres for cancer therapy can be operable in a negative pressure mode in which a negative pressure can be applied to move the suspension of therapeutic radioactive microspheres from the mixing chamber to the microfluidic cassette.

In a twelfth aspect, in addition to one or more of the preceding or following aspects, or in the alternative to some aspects, the fractionation system for microspheres for cancer therapy can be operable in a positive and negative pressure mode in which both a positive and a negative pressure can be applied to move the suspension of therapeutic radioactive microspheres from the mixing chamber to the microfluidic cassette.

In a thirteenth aspect, in addition to one or more of the preceding or following aspects, or in the alternative to some aspects, the system can further include a pump, wherein the pump can be in fluid communication with at least one of a mixing chamber and the microfluidic cassette and can be configured to generate a positive or a negative pressure therein.

In a fourteenth aspect, in addition to one or more of the preceding or following aspects, or in the alternative to some aspects, the microfluidic cassette can be a dual-layer microfluidic cassette.

In a fifteenth aspect, in addition to one or more of the preceding or following aspects, or in the alternative to some aspects, the microfluidic cassette the fractionation system for microspheres for cancer therapy can further include a fluidic layer and a capping layer, wherein the capping layer can be disposed on the fluidic layer.

In a sixteenth aspect, in addition to one or more of the preceding or following aspects, or in the alternative to some aspects, the capping layer can include an embedded structural support.

In a seventeenth aspect, in addition to one or more of the preceding or following aspects, or in the alternative to some aspects, the fractionation system for microspheres for cancer therapy can further include a second microsphere dam, wherein the second microsphere dam can have a physical geometry configured to block microspheres from passing through the microsphere dam, and a second weep channel, wherein the second weep channel can be separated from the microsphere conveying channel by the second microsphere dam.

In an eighteenth aspect, in addition to one or more of the preceding or following aspects, or in the alternative to some aspects, the microsphere dam can include a plurality of segments, wherein at least some of the plurality of segments can be differently sized.

In a nineteenth aspect, in addition to one or more of the preceding or following aspects, or in the alternative to some aspects, wherein segments of a smaller size from amongst the plurality of segments can be oriented towards a proximal end of the microsphere conveying channel.

In a twentieth aspect, in addition to one or more of the preceding or following aspects, or in the alternative to some aspects, the microfluidic cassette the fractionation system for microspheres for cancer therapy can further include at least one sensor. The at least one sensor can include at least one selected from the group consisting of a pressure sensor and a flow rate sensor. The at least one sensor can be configured to measure at least one of a pressure or a fluid flow rate in a channel of the microfluidic cassette.

In a twenty-first aspect, in addition to one or more of the preceding or following aspects, or in the alternative to some aspects, the microsphere dam can be oriented perpendicularly to the microsphere conveying channel.

In a twenty-second aspect, in addition to one or more of the preceding or following aspects, or in the alternative to some aspects, the microsphere dam can be oriented non-perpendicularly to the microsphere conveying channel.

In a twenty-third aspect, in addition to one or more of the preceding or following aspects, or in the alternative to some aspects, the weep channel can be deeper than the microsphere conveying channel.

In a twenty-fourth aspect, in addition to one or more of the preceding or following aspects, or in the alternative to some aspects, the weep channel can be wider than the microsphere conveying channel.

In a twenty-fifth aspect, in addition to one or more of the preceding or following aspects, or in the alternative to some aspects, fluid in the weep channel flows in an opposite direction from the suspension in the microsphere conveying channel.

In a twenty-sixth aspect, a method of fractionating a dose of radioactive microspheres using a microfluidic cassette can be included. The method can include passing a suspension of the radioactive microspheres in a carrier fluid from a mixing chamber into a microsphere conveying channel of the microfluidic cassette, removing carrier fluid from the microsphere conveying channel through at least one microsphere dam into a weep channel, wherein the microsphere dam can be configured to retain the radioactive microspheres, accumulating a predetermined number of microspheres in the microsphere conveying channel, and transferring the microspheres from the microsphere conveying channel to a holding tank by opening a valve.

In a twenty-seventh aspect, in addition to one or more of the preceding or following aspects, or in the alternative to some aspects, the method can further include generating a positive or a negative pressure within the microfluidic cassette to move the suspension of the radioactive microspheres in a carrier fluid therein.

In a twenty-eighth aspect, in addition to one or more of the preceding or following aspects, or in the alternative to some aspects, the method can further include measuring at least one of a pressure or a fluid flow rate in a channel of the microfluidic cassette.

In a twenty-ninth aspect, in addition to one or more of the preceding or following aspects, or in the alternative to some aspects, the amount of radioactive microspheres passed from the mixing chamber to the microsphere conveying channel of the microfluidic cassette as part of one passing operation represents a fraction of the total amount of radioactive microspheres in the mixing chamber.

In a thirtieth aspect, in addition to one or more of the preceding or following aspects, or in the alternative to some aspects, the method can further include returning carrier fluid collected in the weep channel back to the mixing chamber.

In a thirty-first aspect, in addition to one or more of the preceding or following aspects, or in the alternative to some aspects, the method can further include passing carrier fluid collected in the weep channel to a waste container.

In a thirty-second aspect, a fractionation system for microspheres for cancer therapy can be included having a microfluidic cassette. The microfluidic cassette can include a fluidic layer defining a microsphere conveying channel and a capping layer defining a weep channel. The weep channel can be separated from the microsphere conveying channel by a filter layer. The filter layer can define apertures configured to block microspheres from passing through the filter layer. The filter layer can be disposed between the fluidic layer and the capping layer. A fractionation output valve can be included wherein the fractionation output valve can be in fluid communication with the microsphere conveying channel. A weep channel clearance valve can be included wherein the weep channel clearance valve can be in fluid communication with the weep channel. A control circuit can be included wherein the control circuit is configured to control the fractionation output valve and the weep channel clearance valve.

In a thirty-third aspect, in addition to one or more of the preceding or following aspects, or in the alternative to some aspects, the fractionation output valve can be a pneumatically operated valve.

In a thirty-fourth aspect, in addition to one or more of the preceding or following aspects, or in the alternative to some aspects, the weep channel clearance valve can be a pneumatically operated valve.

In a thirty-fifth aspect, in addition to one or more of the preceding or following aspects, or in the alternative to some aspects, the system can further include a holding tank, wherein the holding tank can be in fluid communication with the microsphere conveying channel as interrupted by the fractionation output valve.

In a thirty-sixth aspect, in addition to one or more of the preceding or following aspects, or in the alternative to some aspects, the fractionation system for microspheres for cancer therapy can further include a catheter connection port, and a holding tank output valve, wherein the holding tank can be in fluid communication with the catheter connection port as interrupted by the holding tank output valve.

In a thirty-seventh aspect, in addition to one or more of the preceding or following aspects, or in the alternative to some aspects, the system can further include a plurality of catheter connection ports.

In a thirty-eighth aspect, in addition to one or more of the preceding or following aspects, or in the alternative to some aspects, the system can further include a mixing chamber, wherein the mixing chamber can be configured to supply a suspension of therapeutic radioactive microspheres to the microfluidic cassette.

In a thirty-ninth aspect, in addition to one or more of the preceding or following aspects, or in the alternative to some aspects, the fractionation system for microspheres for cancer therapy can be operable in a positive pressure mode in which a positive pressure can be applied to move the suspension of therapeutic radioactive microspheres from the mixing chamber to the microfluidic cassette.

In a fortieth aspect, in addition to one or more of the preceding or following aspects, or in the alternative to some aspects, the fractionation system for microspheres for cancer therapy can be operable in a negative pressure mode in which a negative pressure can be applied to move the suspension of therapeutic radioactive microspheres from the mixing chamber to the microfluidic cassette.

In a forty-first aspect, in addition to one or more of the preceding or following aspects, or in the alternative to some aspects, the fractionation system for microspheres for cancer therapy can be operable in a positive and negative pressure mode in which both a positive and a negative pressure can be applied to move the suspension of therapeutic radioactive microspheres from the mixing chamber to the microfluidic cassette.

In a forty-second aspect, in addition to one or more of the preceding or following aspects, or in the alternative to some aspects, can further include a pump, wherein the pump can be in fluid communication with at least one of a mixing chamber and the microfluidic cassette and can be configured to generate a positive or a negative pressure therein.

In a forty-third aspect, in addition to one or more of the preceding or following aspects, or in the alternative to some aspects, the microfluidic cassette the fractionation system for microspheres for cancer therapy can further include at least one sensor. The at least one sensor can include at least one selected from the group consisting of a pressure sensor and a flow rate sensor. The at least one sensor can be configured to measure at least one of a pressure or a fluid flow rate in a channel of the microfluidic cassette.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures(FIGS.), in which:

FIG. 1 is a schematic view of a cancer therapy delivery system in accordance with various embodiments herein.

FIG. 2 is a schematic view of components of a cancer therapy delivery system in accordance with various embodiments herein.

FIG. 3 is a schematic sectional view of a microfluidic cassette in accordance with various embodiments herein.

FIG. 4 is a schematic top view of a microfluidic cassette in accordance with various embodiments herein.

FIG. 5 is a sectional view of a microfluidic cassette as taken along line A-A of FIG. 4 in accordance with various embodiments herein.

FIG. 6 is a schematic view of components of a microfluidic cassette in accordance with various embodiments herein.

FIG. 7 is a schematic view of a portion of a microfluidic cassette including a portion of a weep channel in accordance with various embodiments herein.

FIG. 8 is a schematic view of a portion of a microfluidic cassette including a portion of a weep channel in accordance with various embodiments herein.

FIG. 9 is a schematic view of components of a microfluidic cassette in accordance with various embodiments herein.

FIG. 10 is a schematic view of components of a microfluidic cassette in accordance with various embodiments herein.

FIG. 11 is a schematic view of components of a microfluidic cassette in accordance with various embodiments herein.

FIG. 12 is a schematic view of components of a microfluidic cassette in accordance with various embodiments herein.

FIG. 13 is a schematic view of components of a microfluidic cassette in accordance with various embodiments herein.

FIG. 14 is a schematic view of components of a microfluidic cassette in accordance with various embodiments herein.

FIG. 15 is a schematic view of components of a microfluidic cassette in accordance with various embodiments herein.

FIG. 16 is a schematic view of components of a microfluidic cassette in accordance with various embodiments herein.

FIG. 17 is a schematic view of components of a microfluidic cassette in accordance with various embodiments herein.

FIG. 18 is a schematic view of components of a microfluidic cassette in accordance with various embodiments herein.

FIG. 19 is a sectional view of a microfluidic cassette in accordance with various embodiments herein.

FIG. 20 is a schematic view of a filter layer in accordance with various embodiments herein.

FIG. 21 is a schematic view of filter layer variations in accordance with various embodiments herein.

FIG. 22 is a schematic view of a capping layer of a microfluidic cassette in accordance with various embodiments herein.

FIG. 23 is a schematic view of components of a microfluidic cassette in accordance with various embodiments herein.

FIG. 24 is a schematic view of components of a microfluidic cassette in accordance with various embodiments herein.

FIG. 25 is a schematic view of components of a microfluidic cassette in accordance with various embodiments herein.

FIG. 26 is a schematic view of components of a microfluidic cassette in accordance with various embodiments herein.

FIG. 27 is a schematic view of a cancer therapy delivery system in accordance with various embodiments herein.

FIG. 28 is a schematic block diagram of an automated dose fractionation system for radioactive microspheres in accordance with various embodiments described herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, radioactive microspheres have been developed as a means of treating cancer by delivering targeted radiation therapy directly to tumors. Radioactive microspheres can be manufactured, placed in a master vial, and then shipped to a treatment site for clinical use.

In some cases, a clinician may desire to deliver the entire dose of radioactive microspheres in the master vial to a single treatment site during a single infusion event. However, in other cases, a clinician may desire to deliver a fraction of the entire dose of radioactive microspheres and then take an action such as positioning another delivery catheter and/or switching to another delivery catheter and then deliver another fraction or the remainder of the dose within the master vial. Thus, in some cases, there is a need to fractionate the total dose of radioactive microspheres from the master vial into multiple smaller doses. It will be appreciated, however, that there are various challenges associated with the accurate and efficient fractionation of doses.

In accordance with embodiments herein, automated dose fractionation systems for radioactive microspheres using microfluidic cassettes can be utilized in the context of cancer therapy to deliver precise fractionated doses of microspheres containing therapeutic radioisotopes directly to targeted treatment sites. As used herein, the term “microfluidic cassette” shall mean a fluidic device or module that integrates one or more microfluidic circuits such as including fluidic channels into an integrated unit configured for the transport of carrier fluids and/or suspensions of carrier fluids and microspheres as described herein.

Embodiments of fractionation systems herein can include a microfluidic cassette configured with both a microsphere conveying channel and a microsphere dam to achieve controlled, selective retention of radioactive microspheres within the conveying channel while enabling passage of the carrier fluid into a weep channel. The physical geometry of the microsphere dam can include features such as aperture dimensions sized to restrict the transit of microspheres therethrough, supporting accurate accumulation and dose fractionation of the radioactive microspheres within the cassette. As such, by controlling the movement of carrier fluid through the weep channel, the system can facilitate the isolation of a predetermined fraction of microspheres from a bulk suspension.

Integration of controllable valves, such as a fractionation output valve in communication with the microsphere conveying channel and a weep channel clearance valve in communication with the weep channel, enables precise sequential operations. A control circuit can regulate the opening and closing of these valves to coordinate the collection, retention, and transfer of microsphere fractions without user intervention within the cassette’s internal fluidic architecture. This configuration can automate the process of obtaining consistent fractional doses and supports reproducibility across multiple fractionation events.

Systems herein can be configured to operate under multiple different pressure modes, including positive pressure operation, negative pressure operation, or combinations thereof, for directing fluidic movement as required for fractionation and subsequent delivery of the therapeutic suspension.

Advantages of embodiments herein include a reduction in manual handling and cross-contamination risks, as the microspheres are retained and separated within closed microfluidic pathways. Furthermore, by utilizing a microfluidic platform with defined geometric and valving characteristics, the system enables highly reproducible fractionation regardless of how the system is set up and arranged by an individual user. For example, the microfluidic platform eliminates a significant amount of external tubing that otherwise, as arranged by individuals for each procedure, may contribute to variability in fluid resistance, dead volume, and ultimately inconsistency in the amounts of therapeutic microspheres delivered. Thus, embodiments herein provide improved consistency in dose delivery and minimize opportunities for user-introduced error during setup and operation of the system.

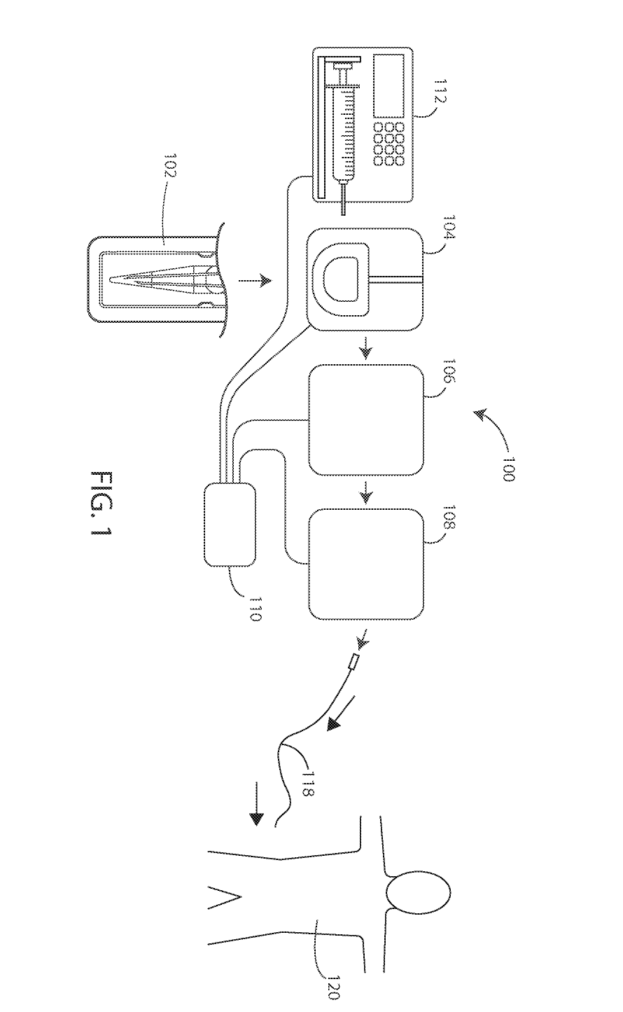

Referring now to FIG. 1, a schematic view of a cancer therapy delivery system 100 is shown in accordance with various embodiments. The system 100 includes a microsphere master supply vial 102 (or master vial), a mixing chamber 104, a fractionation system 106, a holding tank 108, control circuitry 110, and a pump 112. FIG. 1 also depicts a fluid delivery catheter 118 and a patient 120. It will be appreciated that the system 100 can also include other components such as protective shielding that are not shown here for ease of illustration.

The system 100 is responsible for enabling the accurate and controlled fractionation and subsequent transfer of therapeutic radioactive microspheres to the fluid delivery catheter 118 and then into the patient 120 during cancer therapy procedures. Specifically, the system 100 receives a supply of therapeutic radioactive microspheres from a microsphere supply vial 102, directs the microspheres to a mixing chamber 104 for agitation to maintain a suspension of microspheres with a carrier fluid and subsequently transfers the suspension to a fractionation system 106. Within the fractionation system 106, a microfluidic cassette processes the suspension to selectively accumulate a predetermined fraction of microspheres by receiving the suspension and then withdrawing carrier fluid through a weep channel while retaining the microspheres upstream of a microsphere dam. Upon completion of fractionation, the system 100 further enables the transfer of the retained fraction of microspheres from the microfluidic cassette to a holding tank 108. These steps are managed through the coordinated operation of various valves such as output and clearance valves and pumps (such as pump 112) as controlled by a control circuit 110 programmed to regulate fluid flow and microsphere transfer in accordance with predetermined dosing requirements. The system 100 thereby provides for precise, reproducible preparation and delivery of one or more fractionated doses of radioactive microspheres delivered to a patient via a delivery catheter 118, supporting both single-site and multi-site cancer therapy protocols with a reduction in manual intervention and an increase in reproducibility and accuracy.

The flow of the suspension through components of the system can be driven by pressure as provided by one or more pumps. In certain embodiments, the fractionation system 106 for microspheres for cancer therapy can operate in a positive pressure mode, in which a positive pressure is applied to transfer (or push) a suspension of therapeutic radioactive microspheres from the mixing chamber 104 to a microfluidic cassette, and then into a holding tank 108. In other embodiments, the fractionation system 106 can operate in a negative pressure mode, in which a negative pressure is applied to transfer (or pull) the suspension from the mixing chamber 104 to a microfluidic cassette and then into a holding tank 108. In still further embodiments, the fractionation system 106 can operate in a mode utilizing both positive and negative pressures to move the suspension of therapeutic radioactive microspheres from the mixing chamber 104 to the microfluidic cassette and then onto a holding tank 108.

The control circuitry 110 can include various components in order to execute operations as described herein, including causing valves to open and close, controlling pumps (such as pump 112), sending and/or receiving data, receiving instructions from system users, and the like. In various embodiments, the control circuitry 110 can include components such as a microprocessor or microcontroller for executing programmed instructions, input/output (I/O) interfaces for communicating with valve actuators, pump controllers, and sensor modules, non-transitory memory for storing operational programs and control logic, communication interfaces for data exchange with external systems or user interfaces (such as USB, Ethernet, Wi-Fi, or Bluetooth modules), analog-to-digital converters (ADCs) for processing analog sensor signals, digital-to-analog converters (DACs) where required, power management circuitry for supplying and regulating the necessary voltages and currents to circuit elements, and user interface modules such as a touchscreen, push buttons, indicator lights, or audible alarms. In certain embodiments, the control circuitry 110 can also include circuitry configured to perform feedback control based on real-time sensor data, safety interlocks for ensuring proper system operation, and connectors or ports for diagnostic, programming, or maintenance tasks.

Pump 112 is shown adjacent to the mixing chamber 104 and can be used to move the carrier fluid and/or a suspension of microspheres in carrier fluid throughout the cancer therapy delivery system. However, it will be appreciated that pump 112 and, more generally, pumps of the system can be configured to be located at various points and interface with various components of the system herein. In some embodiments, the pump 112 can be used to deliver carrier fluid into the master supply vial 102 through an inlet which then causes a suspension of carrier fluid and microspheres to be pushed out of an outlet and into the mixing chamber 104. In some embodiments, valves can be opened or closed and pump 112 can be used to push a suspension of microspheres and carrier fluid from the mixing chamber 104 and into the fractionation system 106. In some embodiments, pump 112 can also be used to push microspheres from the fractionation system 106 and into the holding tank 108. As described further below, systems herein can operate in positive pressure modes where fluids and/or suspensions are being pushed through the system. However, systems herein can also operate in negative pressure modes where fluids and/or suspensions are being pulled through the system. Systems herein can also operate in mixed positive and negative pressure modes.

In some embodiments, systems herein can include or utilize one, two, three, four, five, or more pumps. Pumps utilized herein can be of various types. By way of example, in some embodiments, pump types herein can include syringe pumps, peristaltic pumps, piston pumps, diaphragm pumps, gear pumps, or vacuum pumps. The selection of pump type can be based on the desired pressure mode (positive or negative), required flow rate, compatibility with radioactive or biological fluids, and integration with the system’s control circuit. In some configurations, a pump can be configured to deliver carrier fluid to a master supply vial or mixing chamber, to transfer a suspension of microspheres and carrier fluid into a microfluidic cassette, or to move fluid between system components such as holding tanks or waste containers. Multiple pumps may be used in a coordinated fashion to enable precise control over the movement of fluids and suspensions for dose fractionation, flushing, or recirculation as applicable to the operational sequence of the cancer therapy delivery system.

In some embodiments, all pumps can be controlled automatically by the system. However, in some embodiments, one or more pumps can be configured to be controlled manually by a system user. By way of example, in some embodiments, a pump used to move a suspension of microspheres and carrier fluid from the holding tank to the fluid delivery catheter can be manually controlled by a system user such as a clinician.

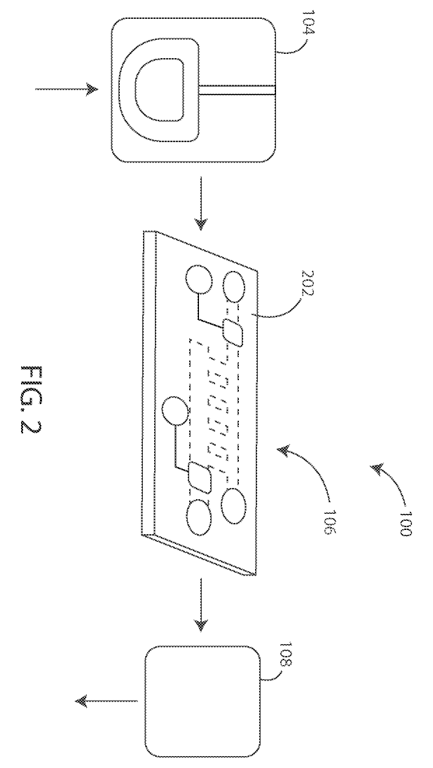

Referring now to FIG. 2, a schematic view of components of a cancer therapy delivery system 100 is shown in accordance with various embodiments. The cancer therapy delivery system 100 includes a fractionation system 106 for microspheres for cancer therapy. The system also includes a mixing chamber 104, a holding tank 108, and a microfluidic cassette 202.

In various embodiments, the microfluidic cassette 202 includes a microsphere conveying channel, a microsphere dam configured to block passage of microspheres while allowing carrier fluid to pass, and a weep channel, with the weep channel being separated from the microsphere conveying channel by the microsphere dam. The microfluidic cassette 202 further includes a fractionation output valve in fluid communication with the microsphere conveying channel, and a weep channel clearance valve in fluid communication with the weep channel. A control circuit is also provided, which is configured to control the operation of the fractionation output valve and the weep channel clearance valve. In some embodiments, the holding tank 108 is in fluid communication with the microsphere conveying channel, with this connection being interrupted or controlled by a fractionation output valve. Additionally, the mixing chamber 104 is configured to supply a suspension of therapeutic radioactive microspheres to the microfluidic cassette 202.

In certain embodiments, the microfluidic cassette 202 is a dual-layer microfluidic cassette. However, other configurations for microfluidic cassettes are also contemplated herein including tri-layer microfluidic cassettes, single-layer microfluidic cassettes, multilayer cassettes containing additional functional layers such as filter layers or sensor layers, cassettes with embedded structural support elements, cassettes having an integrated filter between the fluidic and capping layers, and cassettes fabricated with alternative arrangements of fluidic channels, valves, and structural features as suitable for fractionation, retention, and controlled transfer of radioactive microspheres.

Integration of functional components such as pneumatic or mechanical valves, sensors, or connection ports can be accomplished using embedded inserts, patterned traces, or through-holes fabricated during cassette construction. Connectors can be adapted to interface with fluidic pumps, external reservoirs, delivery catheters, or electronic control modules as set forth in the configurations described elsewhere herein. The material and fabrication methods are selected to ensure compatibility with the thermal, chemical, and radiological environment encountered during clinical preparation, operation, and disposal of the microfluidic cassette within the dose fractionation system for cancer therapy.

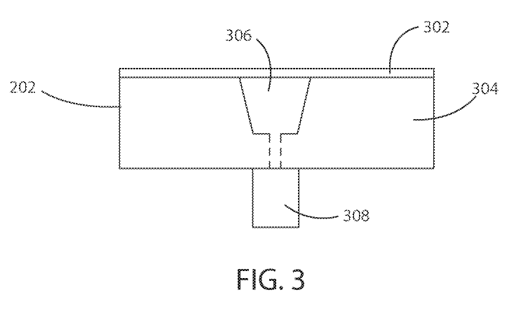

Referring now to FIG. 3, a schematic sectional view of a microfluidic cassette 202 is shown in accordance with various embodiments. The microfluidic cassette 202 includes a capping layer 302 positioned on a fluidic layer 304. Within the fluidic layer 304, a fluid channel 306 is defined. The microfluidic cassette 202 further includes a microfluidic component 308, which can be configured to facilitate fluid handling, microsphere retention, or other process functions as described herein. By way of example, the microfluidic component 308 can be a valve, a pneumatic connection port, a fluidic connection port, a sensor or sensor connector, and/or other components to enable the microfluidic cassette to perform its intended functions.

In various embodiments, the capping layer 302 can be a polymeric sheet. Exemplary polymers can include, but are not limited to, polycarbonate, polymethyl methacrylate (PMMA), polyolefins, and the like. However, other materials for the capping layer 302 can include glasses, ceramics, composites, and the like. The thickness of the capping layer 302 can vary. In some embodiments, the capping layer 302 can be about 0.005, 0.006, 0.007, 0.008, 0.010, or 0.015 inches (0.127 millimeters (mm), 0.152 mm, 0.178 mm, 0.203 mm, 0.254 mm, and 0.381 mm) or greater or less, or an amount falling within a range between any of the foregoing. Generally, thicker capping layers can provide capabilities to operate at higher pressures as they are more resistant to burst or delamination.

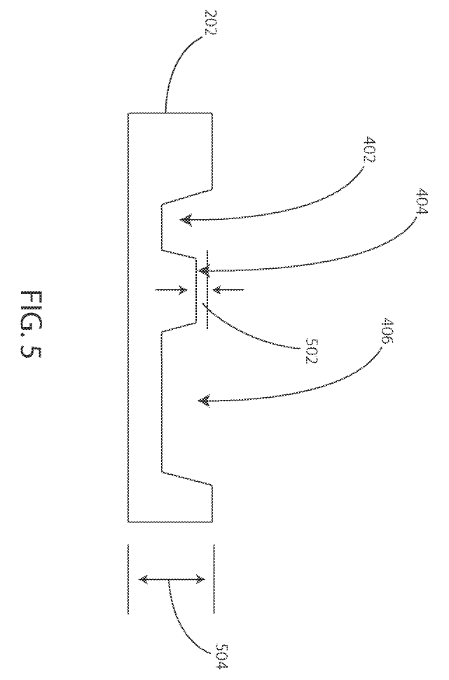

The fluidic layer 304 can also be polymeric. In some embodiments, the fluidic layer 304 can be formed from polymers such as polycarbonate, polymethyl methacrylate (PMMA), polyolefins, and the like. However, other materials for the fluidic layer 304 can include glasses, ceramics, composites, and the like. The thickness of the fluidic layer 304 can vary. Exemplary thickness values for the fluidic layer 304 are described below with respect to FIG. 5.

Fabrication techniques for microfluidic cassettes herein can include deposition printing (3D printing), injection molding, thermoforming, soft lithography, hot embossing, photolithography, laser cutting, laser micromachining, and computer numerical control (CNC) micro-milling. In various embodiments, these fabrication methods can be used individually or in combination to form cassette layers with the required microchannel geometries, weep channels, dams, apertures, sensor interfaces, and other functional features. In certain embodiments, layers manufactured using one or more of these techniques (such as the capping layer 302 and the fluidic layer 304) can be aligned and bonded using thermal, adhesive, or solvent-based bonding processes to form the assembled microfluidic cassette.

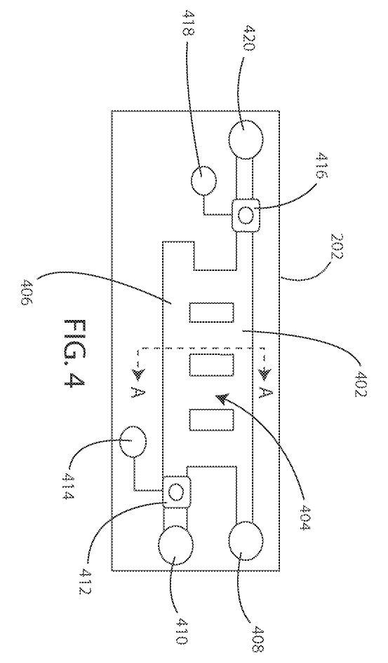

Referring now to FIG. 4, a schematic top view of a microfluidic cassette 202 is shown in accordance with various embodiments. The microfluidic cassette 202, which may be incorporated in a fractionation system for microspheres for cancer therapy, includes a microsphere conveying channel 402, a microsphere dam 404, and a weep channel 406. The microfluidic cassette 202 further includes a mixing chamber input 408, a waste container output 410, a weep channel clearance valve 412, a fractionation output valve 416, and a holding tank output 420. The weep channel clearance valve 412 can be connected to a weep channel clearance pneumatic connection 414 for pneumatic operation of the valve. Similarly, the fractionation output valve 416 can be connected to a fractionation output pneumatic connection 418 for pneumatic operation of the valve.

In various embodiments, the microsphere dam 404 is configured with height, width, and length dimensions such that it allows fluid to pass into the weep channel 406 while physically blocking the passage of microspheres. For example, in some embodiments the microsphere dam 404 has a geometry such as a cross-sectional profile that defines one or more apertures with at least one dimension that is not greater than 20 micrometers such that microspheres of the sizes described herein are physically blocked from passage through the microsphere dam 404. The microsphere dam 404 can be oriented perpendicularly or non-perpendicularly to the microsphere conveying channel 402. The weep channel 406 can be separated from the microsphere conveying channel 402 by the microsphere dam 404 and can be deeper and/or wider than the microsphere conveying channel 402.

In some embodiments, fluid in the weep channel 406 flows in the opposite direction to the suspension in the microsphere conveying channel 402. The weep channel clearance valve 412 is in fluid communication with the weep channel 406. The fractionation output valve 416 is in fluid communication with the microsphere conveying channel 402.

Valves used with microfluidic cassettes herein can be of various types. In some embodiments, valves herein such as the weep channel clearance valve 412, the fractionation output valve 416, as well as other valves herein can be pneumatically operated valves. However, other types of valves are also contemplated herein and can include, but are not limited to, mechanically actuated valves, solenoid-actuated valves, magnetically operated valves, thermally actuated valves, piezoelectrically actuated valves, electrically controlled microvalves, shape-memory alloy actuated valves, and the like. In some embodiments, combination valve assemblies or hybrid actuation approaches can be used to support specific process control needs within the microfluidic cassette.

In operation, the amount of microspheres brought into the microfluidic cassette 202 can be controlled based on controlling the volume of the suspension brought in through the mixing chamber input 408. In some embodiments, the concentration of microspheres in the carrier fluid within the mixing chamber 104 can be a known value based on the starting amount of microspheres in the master supply vial 102 as well as the amount of carrier fluid added resulting in the suspension in the mixing chamber. In this scenario, if further carrier fluid is not added to the mixing chamber as volumes of suspension are passed to the fractionation system 106 (and specifically the microfluidic cassette thereof) then the concentration within the mixing chamber 104 stays constant and therefore the volume of suspension needed to be moved into the fractionation system 106 to transfer a desired amount of microspheres also stays constant. However, if further carrier fluid is added to the mixing chamber 104 as suspension is passed to the fractionation system 106, then the concentration of microspheres in the carrier fluid in the mixing chamber 104 declines according to a dilution curve (like a decay curve) that can be exponential. Since volumes of carrier fluid added to the mixing chamber 104 are known, the dilution curve itself is known. In this scenario, the volume of suspension needed to be moved into the fractionation system 106 to transfer a desired amount of microspheres increases but is a known value based on the dilution curve. As such, the system herein can calculate the volume of suspension that needs to be transferred to transfer a desired amount of microspheres into the fractionation system using the dilution curve.

Referring now to FIG. 5, a sectional view of a microfluidic cassette taken along line A-A of FIG. 4 is shown in accordance with various embodiments herein. The microfluidic cassette includes a microsphere conveying channel 402, a microsphere dam 404, and a weep channel 406. The fluidic layer 304 of the cassette is characterized by a fluidic layer height 504. In some embodiments, the fluidic layer height can be about 0.079 inches (2 millimeters (mm)). However, various other sizes for a fluidic layer are also contemplated herein such as about 0.010 inches (0.25 mm), 0.020 inches (0.5 mm), 0.030 inches (0.75 mm), 0.039 inches (1 mm), 0.049 inches (1.25 mm), 0.059 inches (1.5 mm), 0.069 inches (1.75 mm), 0.079 inches (2 mm), 0.098 inches (2.5 mm), 0.118 inches (3 mm), 0.157 inches (4 mm), 0.197 inches (5 mm), 0.236 inches (6 mm), 0.276 inches (7 mm), 0.315 inches (8 mm), or 0.393 inches (10 mm), or a height falling within a range between any of the foregoing.

In various embodiments, the microsphere dam 404 is dimensioned with a height, width, and length such that it enables carrier fluid to pass from the microsphere conveying channel 402 into the weep channel 406 while blocking the passage of microspheres. In this view, the microsphere dam 404 defines a dam passage height 502. In some embodiments, the dam passage height 502 can be less than the diameter of microspheres used herein in order to block their passage through the dam. In some embodiments, the dam passage height 502 can be less than about 30, 25, 20, 15, 12.5, or 10 micrometers, or can be a height falling within a range between any of the foregoing.

In some embodiments, the weep channel 406 is deeper (has a greater height) than the microsphere conveying channel 402. In some embodiments, the weep channel 406 is wider than the microsphere conveying channel 402.

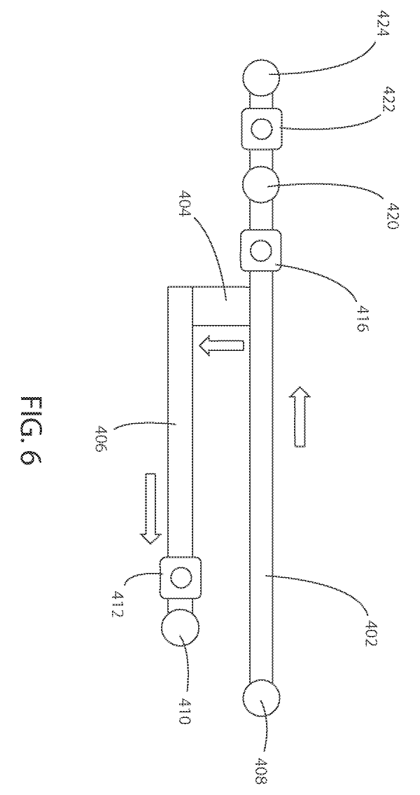

Referring now to FIG. 6, a schematic view of components of a microfluidic cassette is shown in accordance with various embodiments herein. The microfluidic cassette includes a microsphere conveying channel 402, a microsphere dam 404, a weep channel 406, a mixing chamber input 408, a waste container output 410, a weep channel clearance valve 412, a fractionation output valve 416, and a holding tank output 420.

In this embodiment, the fractionation system for microspheres for cancer therapy further includes a holding tank output valve 422 and a catheter connection port 424. The holding tank 108 can be in fluid communication with the catheter connection port 424, with this connection being selectively controlled or interrupted by the holding tank output valve 422. As such, in this embodiment, transit of microspheres from the holding tank 108 to the fluid delivery catheter can be controlled by components on the microfluidic cassette.

In operation, a suspension of microspheres and carrier fluid is transferred from the mixing chamber 104 into the microsphere conveying channel 402 of the microfluidic cassette. During this step, the fractionation output valve 416 is maintained in a closed position, thereby preventing microspheres and carrier fluid from passing into the holding tank 108. This enables the accumulation of a controlled quantity of microspheres within the microsphere conveying channel 402 until the desired number is collected, as determined by the system control circuitry 110. As carrier fluid flows through the microsphere conveying channel 402, it reaches the microsphere dam 404. The microsphere dam 404 is configured to block the passage of microspheres based on its physical geometry, but permits carrier fluid to pass through defined apertures into the weep channel 406. Carrier fluid entering the weep channel 406 is directed, via the weep channel clearance valve 412, through the waste container output 410 and into a waste container (not shown). In some embodiments, rather than direct disposal, the system incorporates a recirculation path (as will be described further below) whereby carrier fluid from the weep channel 406 is routed through a mixing chamber fluid return connection to return to the mixing chamber 104 for reuse.

Once the desired quantity of microspheres has accumulated within the microsphere conveying channel 402 is verified, such as by volumetric control or sensor measurement, the fractionation output valve 416 is actuated to the open position under control of the control circuit 110. This permits transfer of the collected microspheres from the microsphere conveying channel 402 through the holding tank output 420 into the holding tank 108. After the microspheres are received within the holding tank 108, the system may perform a radiation exposure rate measurement using a radiation sensor to confirm that the number of microspheres corresponds to a clinician-specified dose. Following verification, the holding tank output valve 422 can be opened to enable delivery of the microspheres from the holding tank 108 to the catheter connection port 424. The administration of microspheres to the patient via a fluid delivery catheter may be carried out manually, for example by connecting a syringe to the catheter connection port 424 and drawing the microspheres into the catheter, or performed automatically by actuating a syringe pump, peristaltic pump, or other type of pump suitable for providing controlled delivery flow rates. The system thus enables accurate, sequential isolation, verification, and transfer of fractionated doses of radioactive microspheres for clinical administration.

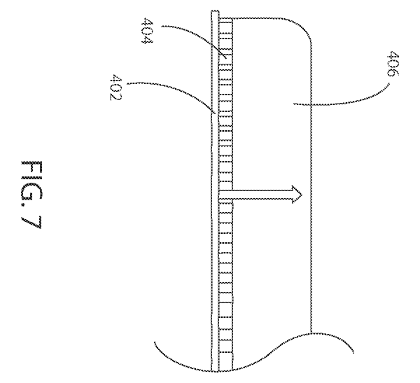

Referring now to FIG. 7, a schematic top view is provided of a portion of the microfluidic cassette illustrating a section of the weep channel 406 in accordance with various embodiments described herein. The microfluidic cassette includes a microsphere conveying channel 402, a microsphere dam 404 disposed between the microsphere conveying channel 402 and a weep channel 406, and the weep channel 406 itself. As shown, the microsphere dam 404 is configured to separate the microsphere conveying channel 402 from the weep channel 406 by allowing carrier fluid to pass through to the weep channel 406 while retaining microspheres within the microsphere conveying channel 402. The depicted arrangement supports the controlled retention and fractionation of microspheres while enabling removal of carrier fluid through the weep channel 406.

In some embodiments, the geometry and sizing of the sphere dam and/or the weep channel can be configured so as promote sufficient fluid flow through the dam while also maintaining structural integrity of the dam and the microfluidic cassette itself.

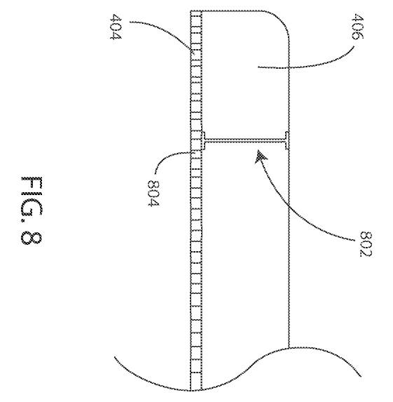

Referring now to FIG. 8, a schematic view of a portion of the microfluidic cassette is shown in accordance with various embodiments herein. FIG. 8 is generally similar to FIG. 7 and shows the weep channel 406 and the microsphere dam 404. As before, the weep channel 406 is separated from the microsphere conveying channel by the microsphere dam 404.

In this view, optional structural columns 804 are shown which can provide structural support in the area of the dam or dams by linking the capping layer and the fluidic layer together. The structural integrity of the sphere dam can be aided by a column size that is sufficient to attach to the capping layer, considering the distance from supporting sections of the fluidic layer. However, limiting the column dimensions allows larger fluid passage areas which promotes further fluid flow through the dam itself.

The width of the weep channel, designated as the weep channel width 802, is also highlighted in this view. The arrangement in FIG. 8 is configured such that the microsphere dam 404 blocks the passage of microspheres while allowing carrier fluid to pass from the microsphere conveying channel into the weep channel 406. The dimensional relationship between the microsphere dam 404 and the weep channel 406, including the specified weep channel width 802, facilitates selective retention of microspheres and removal of carrier fluid as part of the dose fractionation process while not requiring the pressure needed to drive fluid across the dam from being undesirably high. Generally, the larger the weep channel width 802, the less backpressure there is promoting adequate fluid flow through the dam 404. However, a weep channel width 802 that is too large can reduce support for the capping layer and specifically lead to bowing thereof. Therefore, the width can be configured to balance structural support with back pressure concerns. In some embodiments, the weep channel width 802 can be less than about 0.1, 0.08, 0.06, 0.05, 0.04, 0.03, or 0.02 inches (2.54 mm, 2.03 mm, 1.52 mm, 1.27 mm, 1.02 mm, 0.76 mm, and 0.51 mm), or a distance falling within a range between any of the foregoing.

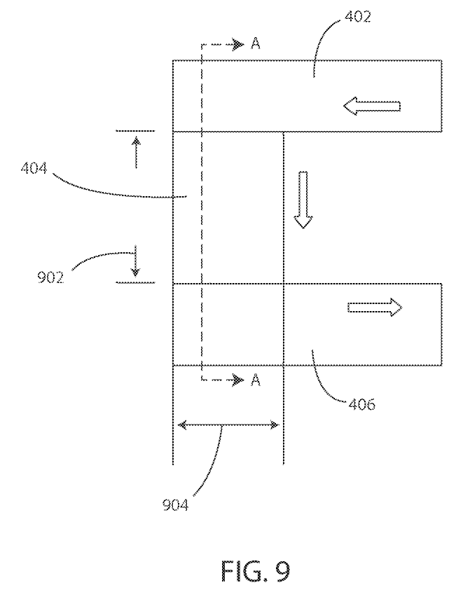

Referring now to FIG. 9, a schematic view of components of a microfluidic cassette is shown in accordance with various embodiments herein. The microfluidic cassette includes a microsphere conveying channel 402, a microsphere dam 404, and a weep channel 406. In this view, the microsphere dam 404 includes a dam separation length 902 and a dam width 904. The dam separation length 902 defines the distance over which the microsphere dam 404 extends between the microsphere conveying channel 402 and the weep channel 406. In general, the larger the dam separation length 902 the larger the backpressure. As such, this distance can be configured to be as small as possible yet sufficient to satisfy other design constraints.

The dam width 904 defines the width of the microsphere dam 404, and specifically the width of a sphere dam channel (there may be multiple dam channels within each dam) parallel to a lengthwise axis of the conveying channel 402. The width 904 can be configured to maximize cross sectional area within the dam or dam channel while also maintaining sufficient delamination strength of the capping layer.

The arrangement of the microsphere conveying channel 402, microsphere dam 404, and weep channel 406 supports selective retention of microspheres within the conveying channel while allowing carrier fluid to pass into the weep channel as part of the dose fractionation process.

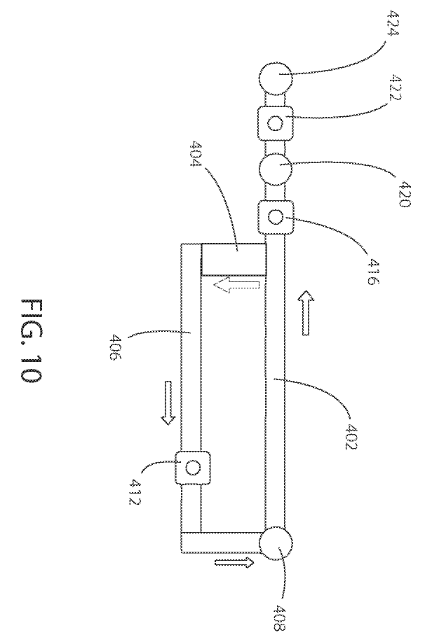

In some embodiments, fluid collected in the weep channel can be returned to the mixing chamber 104 instead of passed to a waste tank. Referring now to FIG. 10, a schematic view of components of a microfluidic cassette is shown in accordance with various embodiments. As before, the microfluidic cassette includes a microsphere conveying channel 402, a microsphere dam 404, and a weep channel 406. The cassette further includes a mixing chamber input 408, a weep channel clearance valve 412, a fractionation output valve 416, a holding tank output 420, a holding tank output valve 422, and a catheter connection port 424. These components are arranged to support the controlled fractionation and transfer of radioactive microspheres during cancer therapy, as described in detail elsewhere herein. However, in this embodiment, the weep channel 406, after the weep channel clearance valve 412, leads back to the mixing chamber via the mixing chamber input 408. In some embodiments, a separate mixing chamber fluid return connection (not shown in this view) can be used to return fluid back to the mixing chamber.

In some embodiments, multiple discrete microsphere dams and/or dam channels can be used to convey fluid from the microsphere conveying channel to the weep channel. Described differently, the microsphere dam can include multiple discrete segments, portions, channels, or apertures. These different dams or segments of a dam can be arranged at different points along the microsphere conveying channel. As the microspheres tend to accumulate near the distal end of the microsphere conveying channel, in some embodiments, the dams or discrete dam segments can be wider or otherwise larger toward the distal end of the microsphere conveying channel such that an accumulation of microspheres will impact the pressure drop of the fluid from the fluid conveying channel to the weep channel less.

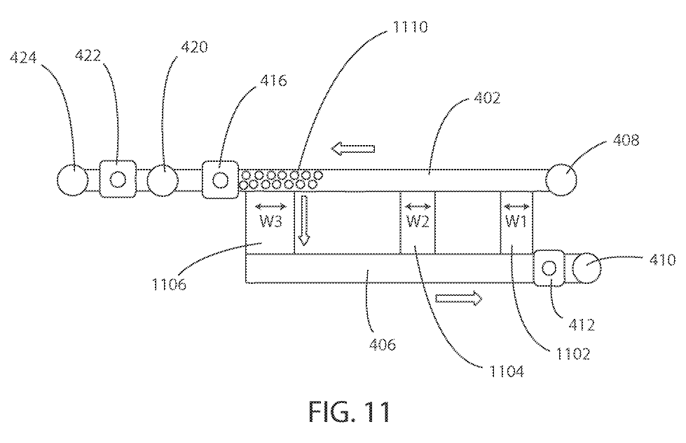

Referring now to FIG. 11, a schematic illustration of components of a microfluidic cassette is provided in accordance with various embodiments herein. As before, the microfluidic cassette includes a microsphere conveying channel 402, a weep channel 406, a mixing chamber input 408, a waste container output 410, a weep channel clearance valve 412, a fractionation output valve 416, a holding tank output 420, a holding tank output valve 422, and a catheter connection port 424.

In this view, accumulated microspheres 1110 are shown within the microsphere conveying channel 402 near a distal end thereof. In the embodiment shown, the microsphere dam 404 includes a plurality of segments, including a first dam segment 1102, a second dam segment 1104, and a third dam segment 1106. These segments can be configured so that at least some have different sizes. In certain embodiments, the segments within the microsphere dam 404 are sized such that smaller segments are located closer to the proximal end of the microsphere conveying channel 402 and larger segments are positioned closer to the distal end of the microsphere conveying channel. This configuration of differently sized segments can be used to optimize microsphere retention and fluid passage through the dam while minimizing pressure increases as microspheres accumulate at the distal end of the microsphere conveying channel.

In some embodiments, the microsphere dam or dams can be oriented substantially perpendicularly to a lengthwise axis of the microsphere conveying channel. However, in other embodiments, at least one microsphere dam can be oriented substantially non-perpendicularly relative to the microsphere conveying channel. For example, the microsphere dam can be oriented at an angle such that it points back in the direction of the proximal end of the microsphere conveying channel (against the direction of flow through the microsphere conveying channel). In some embodiments, the microsphere dam can be oriented at an angle such that it points forward in the direction of the distal end of the microsphere conveying channel (with the direction of flow through the microsphere conveying channel).

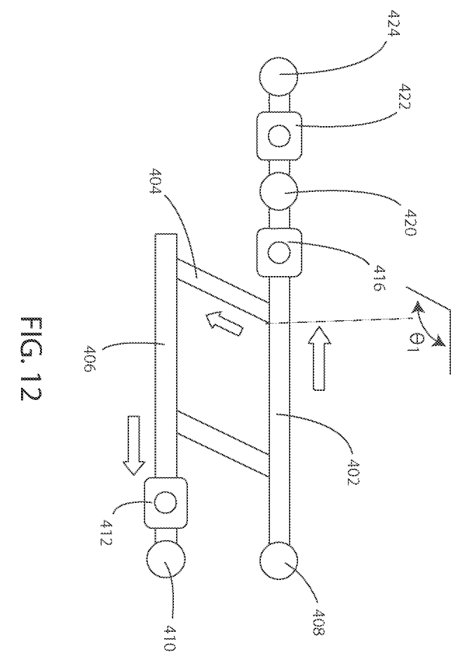

Referring now to FIG. 12, a schematic view of components of a microfluidic cassette is shown in accordance with various embodiments. As before, the microfluidic cassette includes a microsphere conveying channel 402, a microsphere dam 404, and a weep channel 406, a mixing chamber input 408, a waste container output 410, a weep channel clearance valve 412, a fractionation output valve 416, a holding tank output 420, a holding tank output valve 422, and a catheter connection port 424.

In this view, it can be seen that the microsphere dam 404 is oriented at an angle (theta one) that is not perpendicular to the microsphere conveying channel 402. For example, the angle (theta one) can be greater than 90, 100, 110, 120, 130, 140, 150, 160, or 170 degrees, or an angle falling within a range between any of the foregoing.

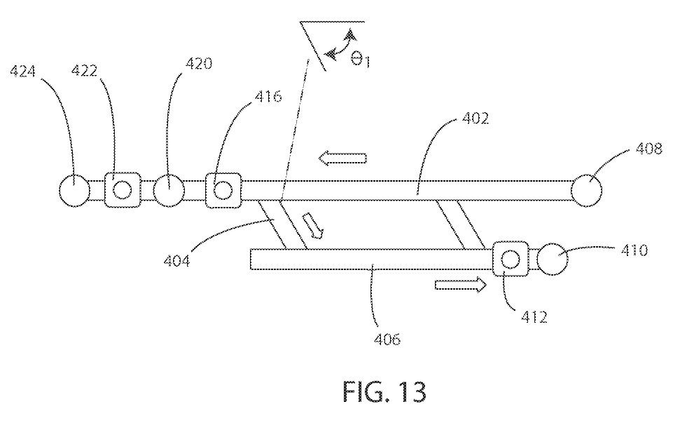

Referring now to FIG. 13, a schematic view of components of a microfluidic cassette is shown in accordance with various embodiments. The microfluidic cassette contains the same components as described with respect to FIG. 12. However, in this view, the microsphere dam 404 is angled with respect to the microsphere conveying channel 402 at a different angle than that shown in FIG. 12. Specifically, in this embodiment, the angle of the microsphere dam 404 with respect to the microsphere conveying channel 402 (theta one) can be less than 90, 80, 70, 60, 50, 40, 30, 20, or even 10 degrees, or an angle falling within a range between any of the foregoing.

In some embodiments, more than one weep channel can be included with the microfluidic cassette per microsphere conveying channel. For example, in some embodiment, a first and a second weep channel can be included and they can be arranged on opposite sides of the microsphere conveying channel.

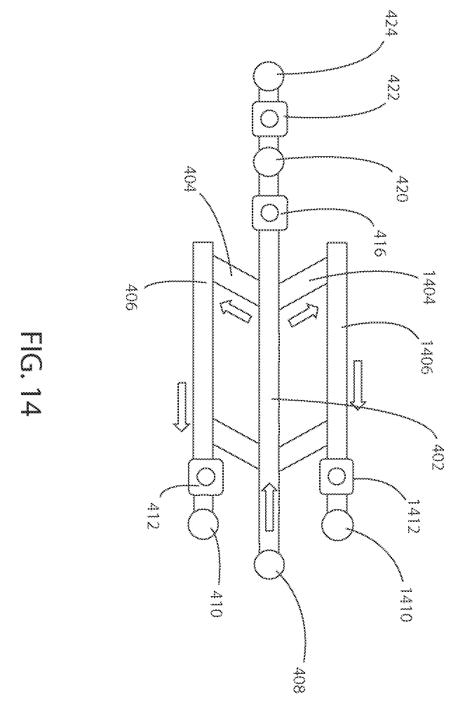

Referring now to FIG. 14, a schematic view of components of a microfluidic cassette is shown in accordance with various embodiments. As before, the microfluidic cassette includes a microsphere conveying channel 402, a microsphere dam 404, a weep channel 406, a mixing chamber input 408, a waste container output 410, a weep channel clearance valve 412, a fractionation output valve 416, a holding tank output 420, a holding tank output valve 422 and a catheter connection port 424.

However, in this embodiment, the microfluidic cassette can also include a second microsphere dam 1404, a second weep channel 1406, a second weep channel clearance valve 1412, and a second waste container output 1410. The second microsphere dam 1404 can have a physical geometry configured to block microspheres from passing through the microsphere dam 404. The second weep channel 1406 can be separated from the microsphere conveying channel 402 by the second microsphere dam 1404. This arrangement allows for additional control in fluid handling and microsphere retention within the microfluidic cassette.

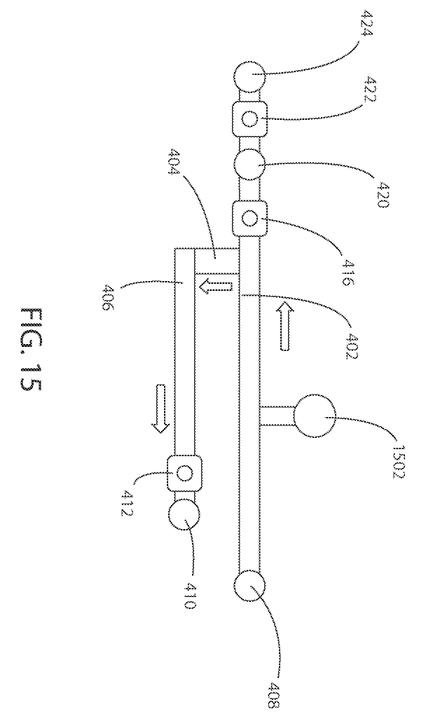

It will be appreciated that various components can be included with microfluidic cassettes. For example, in some embodiments, microfluidic cassettes can include or include ports for one or more sensors. Referring now to FIG. 15, a schematic view of components of a microfluidic cassette is shown in accordance with various embodiments herein. As before, the microfluidic cassette includes a microsphere conveying channel 402, a microsphere dam 404, a weep channel 406, a mixing chamber input 408, a waste container output 410, a weep channel clearance valve 412, a fractionation output valve 416, and a holding tank output 420, a holding tank output valve 422 and a catheter connection port 424.

In this embodiment, the microfluidic cassette further includes at least one sensor, such as a first pressure sensor 1502. The first pressure sensor 1502 can be used to measure the fluid pressure within a designated portion of the microfluidic cassette, such as within the microsphere conveying channel, the weep channel, or another fluidic pathway. In this particular example, the pressure sensor 1502 is configured so that it can measure pressure within the microsphere conveying channel. By monitoring the pressure at these locations, the system is able to detect any rise in pressure that may occur during operation. In this way, the system can monitor pressures within the microfluidic cassette so that it can be controlled and prevented from rising high enough to risk causing damage to the microfluidic cassette.

In some embodiments, the system can monitor pressure values from the pressure sensor 1502 and take various actions depending on values of the same. For example, in some embodiments, the system can be configured to shut off one or more pumps if the pressure exceeds a predetermined threshold value, such as a value that could risk compromising the structural integrity of the microfluidic cassette or safe operation of the system. In some embodiments, a pressure relief valve can be included that automatically opens to relieve excess pressure from the relevant channel or compartment when the measured pressure exceeds a set maximum value, thereby preventing over-pressurization and potential damage to the cassette or associated components.

Other types of sensors beyond pressure sensors are also contemplated herein. In some embodiments, the microfluidic cassette includes at least one sensor selected from the group consisting of a pressure sensor and a flow rate sensor, with the flow rate sensor being configured to measure a fluid flow rate within a channel of the microfluidic cassette. Other types of sensors herein can include, but are not limited to, optical sensors, temperature sensors, conductivity sensors, pH sensors, radiation sensors, and capacitance sensors. These sensors can be configured to monitor relevant parameters within the microfluidic cassette, such as optical density of a suspension, fluid temperature, electrical conductivity or ionic strength, pH level, ionizing radiation emitted by the radioactive microspheres, or the like. In some embodiments the system can also include one or more proximity sensors, such as to detect positioning of components (which could include position of safety shielding or other system components) for safety purposes. Sensor data can be communicated to a control circuit or processing module for system monitoring, feedback control, or data logging as may be required for process management, safety assurance, or quality control within the dose fractionation procedure.

In some embodiments, radiation sensors can be positioned appropriately to measure radiation at various points of the system. By way of example, in some embodiments, a radiation sensor can be positioned to measure radiation at or within the holding tank. In some embodiments, a radiation sensor can be positioned to measure radiation at or within the mixing chamber. Radiation sensors herein can include various types of radiation sensors including, but not limited to, ionization chambers, Geiger-Müller tubes, scintillation detectors, solid-state detectors (such as semiconductor-based detectors), and dosimeter probes.

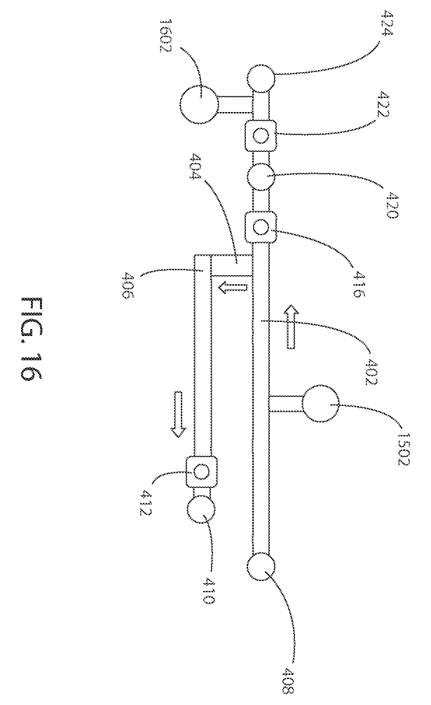

It will be appreciated that in some embodiments multiple sensors can be used. Referring now to FIG. 16, a schematic view of components of a microfluidic cassette is shown in accordance with various embodiments herein. FIG. 16 is similar to FIG. 15 and includes the same components. However, beyond a first pressure sensor 1502, FIG. 16 also includes a second pressure sensor 1602. In this case, the second pressure sensor 1602 is positioned and configured to measure a pressure within the tank output valve 422 and the catheter connection port 424.

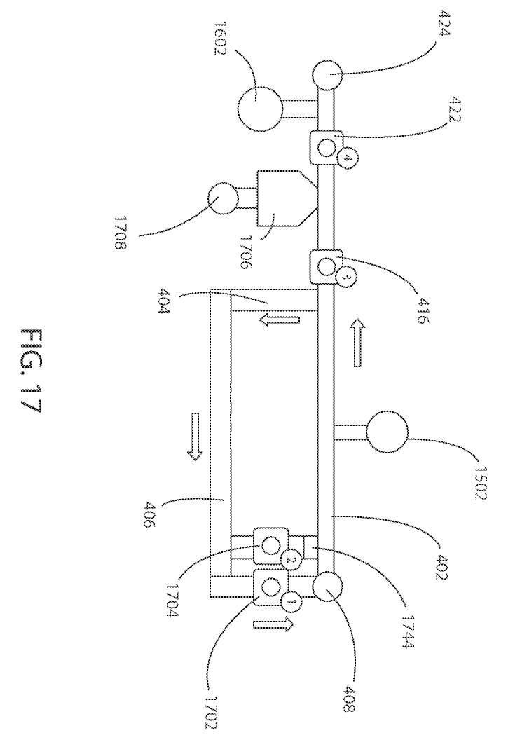

In some cases, clinicians may want to manually control the delivery of microspheres from the holding tank and into the fluid delivery catheter. Systems herein can accommodate such approaches. Referring now to FIG. 17, a schematic view of components of a microfluidic cassette is shown in accordance with various embodiments. The microfluidic cassette includes a microsphere conveying channel 402, a microsphere dam 404, a weep channel 406, a mixing chamber input 408, a fractionation output valve 416, a holding tank output valve 422, a catheter connection port 424, a first pressure sensor 1502, and a second pressure sensor 1602.

To facilitate manual delivery of microspheres from the holding tank, the system depicted in FIG. 17 includes manual administration syringe connection port 1708. In operation, a manually controllable syringe or other pump can be attached to the connection port 1708 and can allow a clinician to manually control the delivery of microspheres from the holding tank and into the fluid delivery catheter.

Holding tanks herein can be separate from the microfluidic cassette or directly integrated therein (an on-cassette holding tank). FIG. 17 schematically illustrates an on-cassette holding tank 1706. The on-cassette holding tank 1706 can be connected downstream of the microsphere conveying channel 402 and arranged to collect fractionated microspheres for subsequent transfer. The holding tank 1706 can be isolated from the fractionation circuit by fractionation output valve 416 and can be isolated from the delivery catheter by tank output valve 422. However, when the tank output valve 422 is opened, then the clinician can administer a dose manually.

In addition, the system illustrated in FIG. 17 further includes a fluid return valve 1702, a bypass flow line valve 1704, and a proximal secondary microsphere dam 1744. The fluid return valve 1702 is configured to selectively permit the return of carrier fluid to a designated subsystem, such as the mixing chamber, thereby supporting recirculation or removal of excess carrier fluid as operationally required. The bypass flow line valve 1704 is disposed to enable a bypass flow pathway to the holding tank 1706 when the microsphere conveying channel 402 has reached its intended filling condition with microspheres. In this operational state, it is desirable to prevent additional fluid from being conveyed from the mixing chamber or from upstream valves, such as the weep channel clearance valve 412 and the mixing chamber input 408, to avoid unintentional introduction of further microspheres into the microsphere conveying channel 402. Accordingly, once the desired quantity of microspheres has been accumulated, the bypass flow line valve 1704 is actuated to the open state. Simultaneously, the fractionation output valve 416 is opened, providing a pressurized or flow-driven pathway for carrier fluid to transfer the collected microspheres from the microsphere conveying channel 402 into the on-cassette holding tank 1706.

In some embodiments, proximal secondary microsphere dam 1744 can be included to retain microspheres within the microsphere conveying channel 402 yet allow fluid to pass into the weep channel 406. The fluid return valve 1702 can be configured to allow carrier fluid to be returned to a selected location, such as the mixing chamber, as desired.

In operation, the system can assume different operational states or operation phases and valves of the system can be opened or closed to facilitate the same. In a phase where microspheres are being passed into or loaded into the microsphere conveying channel, then valve 1702 can be open, valve 1704 can be closed, valve 416 can be closed, and valve 422 can be closed. In a phase where microspheres are being transferred from the microsphere conveying channel 402 to the holding tank 1706, then valve 1702 can be closed, valve 1704 can be open, valve 416 can be open, and valve 422 can be closed. In a phase where microspheres are being transferred from the holding tank 1706 to the fluid delivery catheter, then valve 1702 can be closed, valve 1704 can be closed, valve 416 can be closed, and valve 422 can be open.

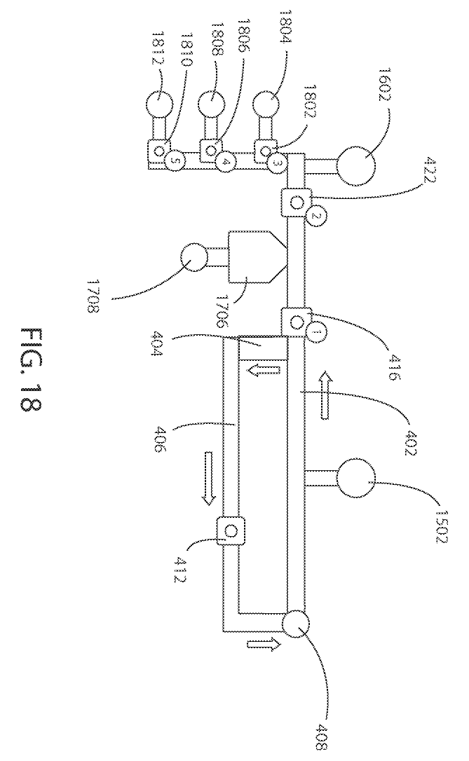

In various embodiments, the system can include a plurality of catheter connection ports. This can allow the system to selectively route microspheres herein into one of a plurality of different fluid delivery catheters as desired by a clinician. Referring now to FIG. 18, a schematic view of components of a microfluidic cassette is shown in accordance with various embodiments herein. As before, the microfluidic cassette includes a microsphere conveying channel 402, a microsphere dam 404, and a weep channel 406, a mixing chamber input 408, a weep channel clearance valve 412, a fractionation output valve 416, a holding tank output valve 422, a first pressure sensor 1502, a second pressure sensor 1602, an on-cassette holding tank 1706, and a manual administration syringe connection port 1708.

In this embodiment, the microfluidic cassette also includes a plurality of catheter output valves, including a first catheter output valve 1802, a second catheter output valve 1806, and a third catheter output valve 1810. Similarly, the system includes a plurality of catheter connection ports, specifically including a first catheter connection port 1804, a second catheter connection port 1808, and a third catheter connection port 1812, arranged to connect to the corresponding catheter output valves. The inclusion of multiple catheter output valves and catheter connection ports facilitates the delivery of fractionated doses through separate catheter pathways as required by clinical protocols without a need for disconnecting and reconnecting catheters from the system herein. This can provide benefits such as reducing the risk of cross-contamination between delivered fractions, minimizing procedure time by eliminating manual disconnection steps, and supporting sequential dosing to multiple target sites during a single treatment session.

Many different configurations for microfluidic cassettes are contemplated herein. In some embodiments, the capping layer can define (in full or in part) one or more channels herein such as the microsphere conveying channel and/or the weep channel.

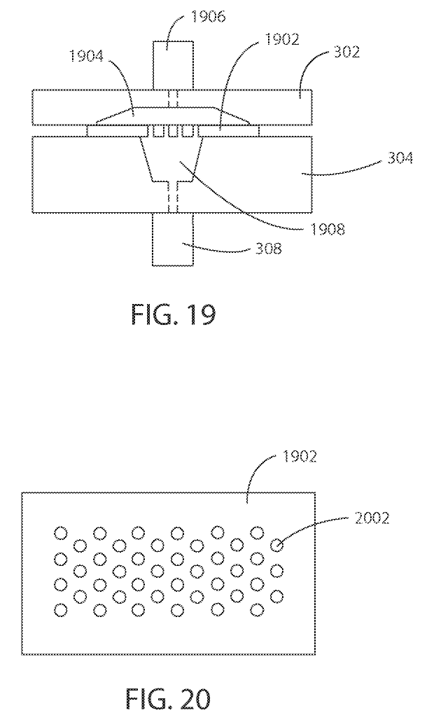

Referring now to FIG. 19, a sectional view of a portion of a microfluidic cassette is shown in accordance with various embodiments. The microfluidic cassette includes a capping layer 302 that defines a cap layer weep channel 1904. The cap layer weep channel 1904 can be in addition to or in place of the arrangement of weep channels as described with respect to other embodiments herein. In such an embodiment, the capping layer 302 can be thicker than in other embodiments described herein in order to accommodate the cap layer weep channel 1904. In some embodiments, the capping layer 302 can be from 0.020 inches to 0.236 inches (0.5 mm to 6 mm) thick. The cassette can further include a fluidic layer 304 defining a microsphere conveying channel 1908, a microfluidic component 308, and top surface fluidic component 1906.

In this embodiment, a filter layer 1902 can be included. The filter layer 1902 can be disposed between the fluidic layer 304 and the capping layer 302. The filter layer 1902 can define one or more apertures configured to block microspheres from passing through the filter layer 1902, thereby retaining microspheres within the microsphere conveying channel 1908 while permitting the carrier fluid to pass. The apertures can be less than about 30, 20, 15, or even 10 micrometers in diameter in order to effectively block the passage of microspheres herein.

Various materials can be used to construct the filter layer 1902 including, but not limited to, polymeric materials such as polycarbonate, polymethyl methacrylate (PMMA), polyolefins, polyethylene terephthalate (PET), or polytetrafluoroethylene (PTFE); metallic materials such as stainless steel, nickel, or titanium; ceramic materials such as alumina or silica; glass materials; or composite materials including combinations of polymers, metals, ceramics, or glasses. In some embodiments, the filter layer 1902 may be formed from a porous membrane, a perforated thin sheet, a micromachined structure, or a woven or non-woven mesh provided the material structure allows for the required fluid permeability and effective microsphere retention.

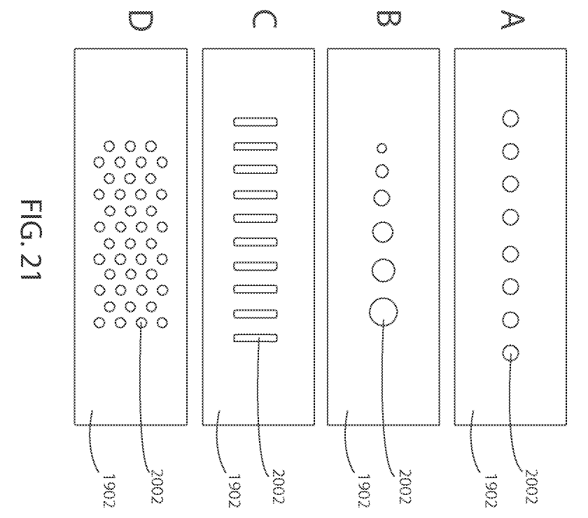

Referring now to FIG. 20, a schematic view of a filter layer 1902 is shown in accordance with various embodiments herein. The filter layer 1902 defines at least one aperture 2002. The filter layer 1902 is configured such that the aperture 2002 prevents microspheres from passing through the filter layer 1902, while allowing carrier fluid to pass. The size and arrangement of the aperture(s) 2002 within the filter layer 1902 are selected to ensure effective retention of microspheres during fractionation while permitting passage of the carrier fluid as part of the dose fractionation process.

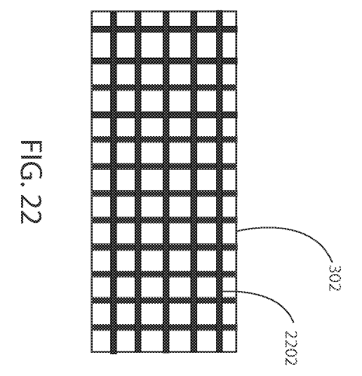

It will be appreciated that apertures in the filter layer can take on many different shapes and configurations. FIG. 21 shows four different examples (A-D) of apertures 2002 within filter layers 1902 herein. The size, shape, and arrangement of apertures 2002 in the filter layer 1902 can be modified to achieve the desired balance of retaining microspheres and allowing passage of carrier fluid, in accordance with the requirements of the fractionation processes described herein. The apertures of filter layers herein can be formed in many different shapes, including, but not limited to, circular, oblong, polygonal (such as rectangular, square, or hexagonal), barred, or slit-like shapes. In some embodiments, the distribution of apertures along the filter layer can be substantially symmetrical from side to side and/or from top to bottom, but in other embodiments, the distribution can be asymmetrical, with more apertures disposed toward one side or another.

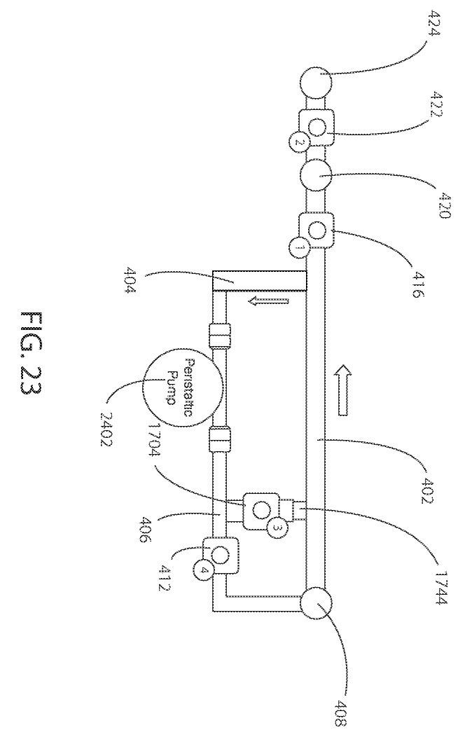

In some embodiments, one or more structural supports can be included to provide support for the capping layer in order to prevent it from bowing or otherwise physically distorting in response to pressures applied herein. Referring now to FIG. 22, a schematic view of a capping layer of a microfluidic cassette is shown in accordance with various embodiments. In some embodiments, the capping layer 302 includes an embedded structural support 2202. The embedded structural support 2202 may enhance the mechanical stability, integrity, and/or rigidity of the capping layer 302 during operation of the microfluidic cassette. The structural support 2202 can take many different forms. By way of example, the embedded structural support 2202 can include a grid, mesh, rib, lattice, or web-like structure formed from a material such as polymer, metal, or composite. In some embodiments, the structural support 2202 can be integrated in-plane with the capping layer, molded or bonded therein, or disposed as a separate insert within the capping layer 302. The structural support 2202 can be configured to span a portion or the entirety of the capping layer 302, and may define apertures or openings as needed to permit formation of channels or ports therethrough. In some embodiments, the embedded structural support 2202 helps reduce deflection or bowing of the capping layer 302 in response to fluid pressures applied during fractionation and transfer of microspheres, thereby maintaining precise alignment and consistent channel geometry throughout the operational cycle of the system.

As previously referenced herein, the system can operate in positive pressure, negative pressure, or mixed-pressure modes. By way of example, in a positive pressure mode, a pump can be coupled upstream of the microfluidic cassette, such as coupled to the mixing chamber or to an upstream port of the microfluidic cassette to pump carrier fluid and generate a positive pressure sufficient to drive the suspension of radioactive microspheres through the fluidic channels of the microfluidic cassette. The mixing chamber can be full of liquid and configured to be rigid such that carrier fluid provided by the pump will not simply cause the mixing chamber to expand or compress a headspace gas. Rather, in this mode, the carrier fluid and suspended microspheres are pushed out of the mixing chamber and into the microfluidic cassette and, specifically, through the conveying channel until a desired quantity accumulates upstream of the microsphere dam, with carrier fluid being forced through the dam and into the weep channel for extraction or recirculation.

In a negative pressure mode, a vacuum source or suction pump can be connected to a downstream port, such as at or adjacent to the holding tank or waste container, thereby generating a negative gauge pressure within the microfluidic cassette and pulling the suspension from the mixing chamber through the cassette by suction. The negative pressure induces flow of the suspension through the conveying channel while enabling removal of carrier fluid via the weep channel, again retaining the microspheres upstream of the dam.

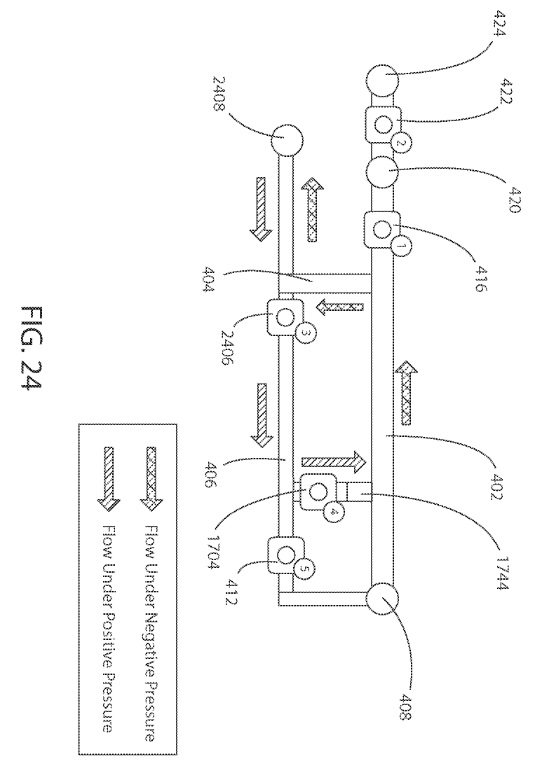

Referring now to FIG. 23, a schematic view of components of a microfluidic cassette is shown in accordance with various embodiments. The microfluidic cassette includes a microsphere conveying channel 402, a microsphere dam 404, a weep channel 406, a mixing chamber input 408, a weep channel clearance valve 412, a fractionation output valve 416, and a holding tank output 420. The fractionation system for microspheres for cancer therapy further includes a holding tank output valve 422, a catheter connection port 424, dam valve 1704, and secondary microsphere dam 1744.

In various embodiments, the system can further include a pump, such as the peristaltic pump 2402, which is in fluid communication with at least one of the mixing chamber 104 and the microfluidic cassette. The pump is configured to generate a negative pressure within the relevant fluidic channels to facilitate movement of the suspension of microspheres and carrier fluid as described herein.

In operation, during a phase of passing microspheres from the mixing chamber into the microsphere conveying channel 402, valve 416, valve 422, and valve 1704 can be closed, while valve 412 can be open. Once the microsphere conveying channel 402 contains the desired number of microspheres, then in a phase of passing microspheres from the microsphere conveying channel 402 into the holding tank, then the valve 416 and valve 1704 can be open while valve 422 and valve 412 can be closed. Then, in a phase of transferring the microspheres from the holding tank into a fluid delivery catheter, valve 416, valve 1704, and valve 412 can be closed while valve 422 can be open.

In some embodiments, a combination of positive and negative pressure is applied. For example a positive pressure source can act at the mixing chamber to propel the suspension forward and a concurrent negative pressure can be applied at a downstream location (e.g., holding tank or waste container) to further enhance flow control, reduce the required pressure gradient, or precisely regulate the rate and direction of microsphere and fluid movement through the cassette. The sources of positive pressure and negative pressure can both be pumps and can be operated at the same rate so that the system does not experience a net rising pressure or falling pressure between the sources of the positive and negative pressure. The capability to switch between or simultaneously use positive and negative pressure provides flexibility so that the operational parameters can be tailored to accommodate clinical protocol requirements, as well as to facilitate flushing, clearing, or recirculating carrier fluids as needed for accurate dose fractionation and delivery.

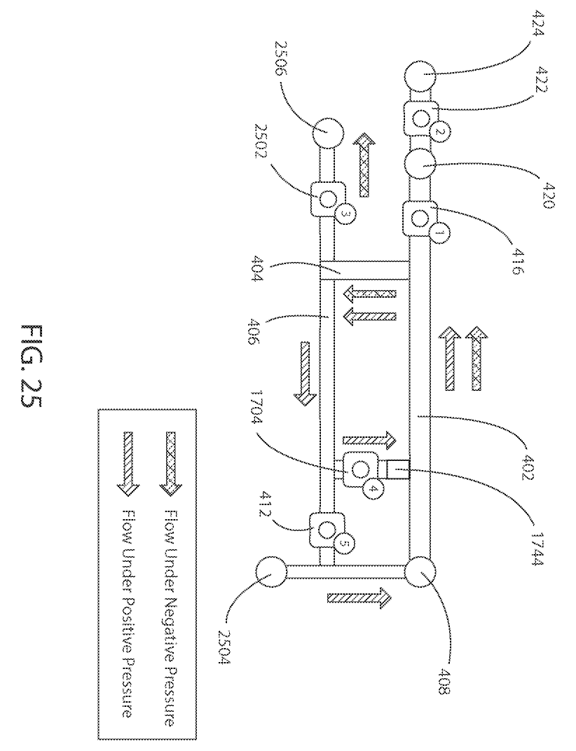

In some embodiments, a dual-direction pressure source can be utilized that can operate as a source of positive pressure at some times and as a source of negative pressure at other times. Referring now to FIG. 24, a schematic view of the components of a microfluidic cassette is shown in accordance with various embodiments. As before, the microfluidic cassette includes a microsphere conveying channel 402, a microsphere dam 404, a weep channel 406, a mixing chamber input 408, a weep channel clearance valve 412, a fractionation output valve 416, a holding tank output 420, a holding tank output valve 422 and a catheter connection port 424, dam valve 1704, and secondary microsphere dam 1744. In this embodiment, the system can also include a pressure direction control valve 2406 and a dual-direction pressure source connection 2408.

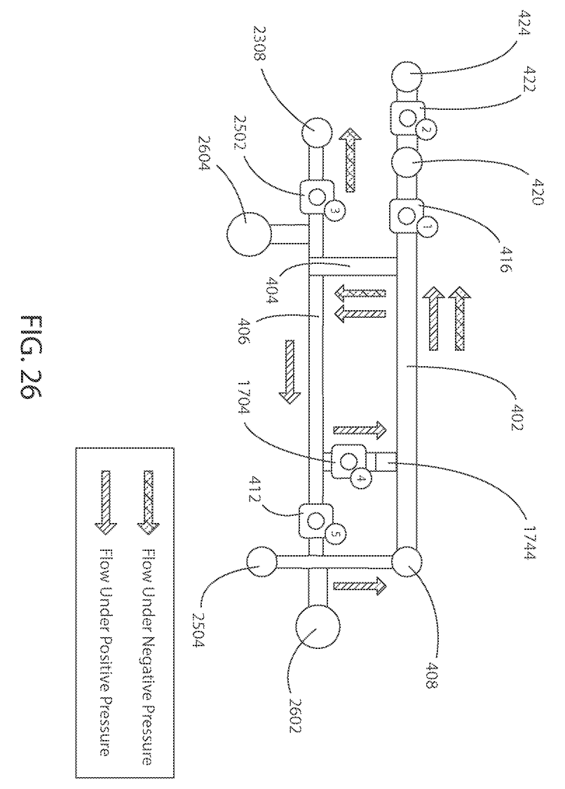

In some embodiments, separate positive pressure sources and negative pressure sources can be utilized that can, at times, operate simultaneously and cooperatively. Referring now to FIG. 25, a schematic view of components of a microfluidic cassette is shown in accordance with various embodiments. The microfluidic cassette includes a microsphere conveying channel 402, a microsphere dam 404, a weep channel 406, and a mixing chamber input 408, a weep channel clearance valve 412, a fractionation output valve 416, a holding tank output 420, holding tank output valve 422, catheter connection port 424, dam valve 1704, and secondary microsphere dam 1744. The microfluidic cassette can also include a vacuum rated valve 2502, a positive pressure source 2504, and a negative pressure source 2506. This configuration is arranged to support operations involving both positive and negative pressure delivery modes, precise retention and fractionation of microspheres, and controlled routing of carrier fluid and microspheres through the system as described herein.

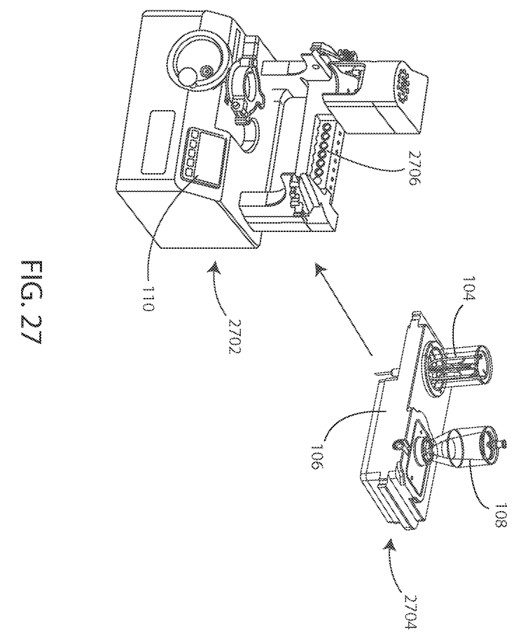

In some embodiments, the system can include pressure sensors along with positive and negative pressure feedback loops to operate the system herein. This can be advantageous as an unexpected pressure rise in pressure within the microsphere conveying channel or in the weep channel would be detected and a system control algorithm can be applied by the control circuit of the system to modulate the positive pressure source to lower the rate of positive pressure generation to allow pressure decay in the system and minimize the risk of chip delamination or failure. Referring now to FIG. 26, a schematic diagram of components of a microfluidic cassette is shown in accordance with various embodiments. The components shown in FIG. 26 are generally similar to those shown in FIG. 25. However, FIG. 26 also shows a first pressure control sensor 2602 and a second pressure control sensor 2604, each configured to monitor pressure at selected locations within the fluidic network.

In operation, during a phase of passing microspheres from the mixing chamber into the microsphere conveying channel 402, valve 416 can be closed, valve 422 can be closed, valve 2502 can be open, valve 1704 can be open, and valve 412 can be closed. The positive pressure source 2504 can be on and controlled by feedback from the first pressure control sensor 2602 and the second pressure control sensor 2604 during this phase. Similarly, the negative pressure source 2308 can be on and controlled by feedback from the first pressure control sensor 2602 and the second pressure control sensor 2604 during this phase. During a second phase of transferring microspheres from the microsphere conveying channel 402 to the holding tank, valve 416 can be open, valve 422 can be closed, valve 2502 can be closed, valve 1704 can be open, and valve 412 can be open. During this phase, the positive pressure source 2504 can be on and controlled by feedback from the first pressure control sensor 2602 and the second pressure control sensor 2604, but the negative pressure source 2308 can be turned off. During a third phase of transferring microspheres from the holding tank to a fluid delivery catheter, valve 416 can be closed, valve 422 can be open, valve 2502 can be closed, valve 1704 can be closed, and valve 412 can be closed. During this phase, the positive pressure source 2504 and the negative pressure source 2308 can be turned off and microspheres can pass out of the holding tank 420 through the catheter connection port 424 and into the fluid delivery catheter via gravity and/or via a manually controlled pump such as a syringe pump.

In some embodiments, certain components of the system can be disposable while other components can be reused. By way of example, portions of the system that contact radioactive microspheres can be configured to be disposable while other portions (control hardware, pumps, etc.) can be reused.

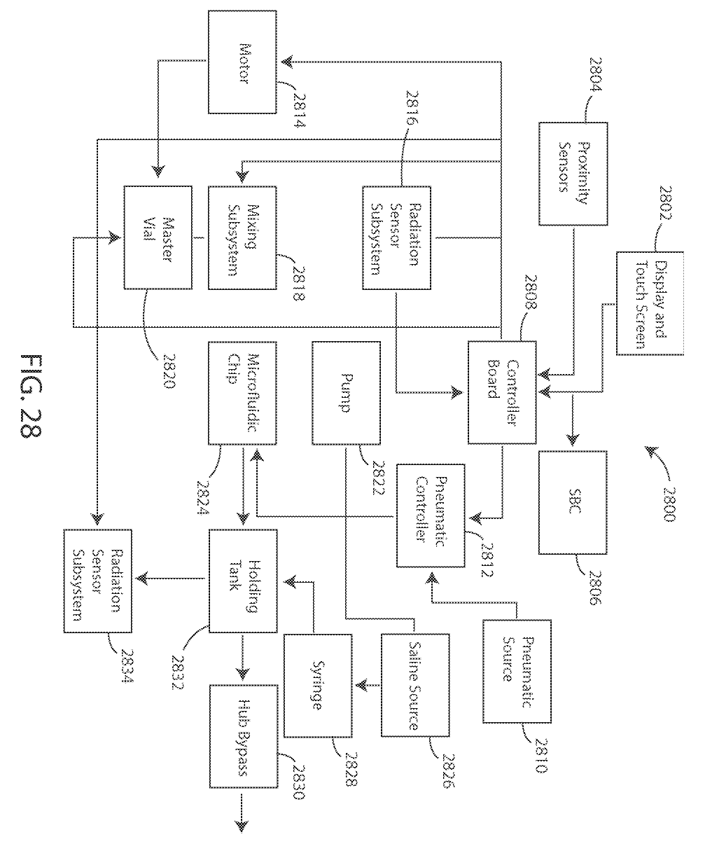

Referring now to FIG. 27, a schematic view is shown of a cancer therapy delivery system in accordance with various embodiments herein. The system can include mixing chamber 104, fractionation system 106, holding tank 108, and control circuitry 110. This view also shows a reusable portion 2702 and a disposable portion 2704. Notably, the mixing chamber 104, fractionation system 106, and holding tank 108 are a part of the disposable portion 2704. In addition, the control circuitry 110 and pneumatic control inputs 2706 (for controlling pneumatically actuated valves) are part of the reusable portion 2702. Other components, not shown in this view, that can be a part of the reusable portion 2702 include components that do not contact radioactive microspheres such as pumps, pressure regulators, electronic control modules, pneumatic supply lines, and electrical interface connections. In contrast, the disposable portion 2704 can include any component or assembly intended to come into contact with radioactive microspheres, such as a microfluidic cassette, fluidic connectors, fluidic tubing, single-use valves, or any integrated sensors that are not re-used between patients.

In some embodiments, the disposable portion 2704 can snap onto the reusable portion 2702 or otherwise be secured thereto for purposes of delivering microspheres to a patient herein. Then, after the procedure has been completed, the disposable portion 2704 can be removed from the reusable portion 2702 and disposed of appropriately.

Referring now to FIG. 28, a schematic block diagram depicts an embodiment of an automated fractionation system 2800 for radioactive microspheres in accordance with the disclosure provided herein. The system 2800 includes a user interface 2802, which includes a graphical display and, in some embodiments, a touchscreen input module configured to receive operator commands and display status notifications associated with system operation. The user interface 2802 is operatively coupled to a system control circuit 2808, which can comprise a microprocessor or microcontroller programmed to coordinate the operation of interconnected subsystems, execute preconfigured or operator-initiated workflow instructions, and enforce established process safety protocols.

As further shown in FIG. 28, the system 2800 can include one or more presence or proximity sensors 2804, each operatively linked to the system control circuit 2808 and configured to detect the presence, position, or correct engagement of selected system components. These sensors 2804 may be configured to confirm that assemblies such as a master microsphere vial receiver or mixing chamber are correctly loaded and secured before enabling active fluidic operations or releasing electronic safety interlocks. In some embodiments, a motor drive 2814 may be included and arranged to raise or lower a platform or support to position the master vial for proper fluidic connection with a mixing subsystem 2818.

The control circuit 2808 can be further coupled to, or incorporate, a local processing module such as a single-board computer (SBC) 2806 which may execute logic for time-based valve actuation, process step sequencing, and provide network communications or remote monitoring capabilities as desired. In various embodiments, the system 2800 includes a pneumatic pressure source 2810 together with a pressure regulator or controller module 2812, each operably managed by the system control circuit 2808. The pneumatic subsystem functions to supply pressurized gas for the actuation of pneumatically-controlled valves or other fluidic components including the fractionation platform. A carrier fluid supply 2826 (e.g., sterile saline) is also provided, with fluid transfer into the system performed by a pump, such as a syringe drive 2828, under control of the system.

A master vial 2820 containing therapeutic radioactive microspheres is connected to the system, and is in operative fluid communication with a mixing subsystem 2818 which includes a mixing container as described elsewhere herein. The system 2800 effects the controlled transfer of microspheres and carrier fluid from the master vial 2820 to the mixing subsystem 2818, using coordinated actuation of pumps and/or pneumatic flow paths. The mixing subsystem 2818 includes an electronically-controlled stirring or agitation mechanism configured to maintain homogeneity of the microsphere suspension within the mixing container by rotating or reciprocating an impeller, paddle, or other suitable agitating structure.

Integrated with the mixing subsystem 2818 is a radiation sensor assembly 2816, configured to monitor the radioactive content of the mixing container to enable validation of process steps, including confirmation that the intended dose or quantity of microspheres has been successfully transferred from the master vial 2820, and to support automated or semi-automated safety checks based on sensor feedback.

Upon completion of mixing, an automated pump 2822, controlled by the system control circuit 2808, is actuated to transfer a predetermined volume of the microsphere suspension from the mixing subsystem 2818 into a microfluidic subsystem 2824. The microfluidic subsystem 2824 can comprise one or more microfluidic cassettes or cartridges as disclosed elsewhere herein and is configured for precision fractionation of the suspension to sharply defined dose sizes. Following fractionation within the microfluidic subsystem 2824, the selected dose or fraction is delivered into a holding vessel 2832. In certain embodiments, the holding vessel 2832, which can be formed as a temporary holding reservoir disposed between the mixing subsystem 2818 and a fluid delivery catheter, is equipped with a radiation sensor 2834 for independent, quantitative verification of the radioactivity of the prepared dose.

The system 2800 may also include, downstream of the holding vessel 2832, a connector hub or bypass module 2830 arranged to minimize fluidic dead volume at the interface with a fluid delivery catheter, thereby promoting accurate transfer of the dose to the patient.

Throughout the described processing pathway, the management and coordination of all pumps, valves, sensors, motors, interlocks, and fluidic processes is performed by the central system control circuit 2808, which is programmed to generate, receive, and respond to process control signals (e.g., sensor readings, valve actuator states, pneumatic pressures, and system alerts) according to programmed control algorithms or operator instructions. In this manner, the system provides a closed-loop operational framework supporting preconfigured, operator-initiated, or sensor-triggered workflows for the secure and reproducible fractionation, verification, and transfer of radioactive microsphere dose fractions for therapeutic use.

### Methods

Various methods are contemplated herein, including methods of manufacture, methods of use, methods of fractionating microspheres, methods of delivering microsphere therapy to a patient and related processes. The operational aspects of the systems and devices described elsewhere in this application can be performed as operations or steps of one or more methods in accordance with the embodiments disclosed.

In some embodiments, one or more of the steps or operations described can be performed as part of a computer-implemented method, executed by one or more processors of one or more computing devices. In these embodiments, instructions for performing the operations may be stored on a non-transitory, computer-readable medium and, when executed by one or more processors, cause the method steps or operations to be carried out by the system.

In one embodiment, a method of fractionating a dose of radioactive microspheres using a microfluidic cassette is provided. The method can include passing a suspension of the radioactive microspheres in a carrier fluid from a mixing chamber into a microsphere conveying channel of the microfluidic cassette, removing carrier fluid from the conveying channel through at least one microsphere dam into a weep channel, where the microsphere dam is configured to retain the radioactive microspheres, accumulating a predetermined number of microspheres in the conveying channel, and then transferring the microspheres from the conveying channel to a holding tank by opening a valve.

In some embodiments, the method further includes generating a positive or negative pressure within the microfluidic cassette to move the suspension of radioactive microspheres in the carrier fluid.

In some embodiments, the method further includes measuring at least one of pressure or fluid flow rate in a channel of the microfluidic cassette.

In certain embodiments, the amount of radioactive microspheres passed from the mixing chamber into the microsphere conveying channel during one passing operation constitutes a fraction of the total amount of radioactive microspheres in the mixing chamber.

In some embodiments, the method further includes returning carrier fluid collected in the weep channel back to the mixing chamber. In some embodiments, the method further includes transferring carrier fluid collected in the weep channel to a waste container.

In additional embodiments, methods are provided for sequential fractionation and delivery of multiple dosage fractions of radioactive microspheres from a single microfluidic cassette within one treatment session. Such methods can include one or more operations of introducing a suspension of radioactive microspheres from a primary vial or mixing chamber into the microfluidic cassette; isolating a first fraction of microspheres within a designated portion of the microsphere conveying channel via an actuation sequence of the weep channel clearance valve and the fractionation output valve so that a predetermined number of microspheres accumulate upstream of the microsphere dam, transferring the isolated first fraction into a holding tank by actuating the fractionation output valve, and transferring the microspheres from the holding tank to a fluid delivery catheter. Various of these operations can be repeated in order to transfer additional fractions of microspheres into the same fluid delivery catheter or a separate fluid delivery catheter. In specific embodiments, pressure or flow sensors can be monitored during each cycle to verify proper operation of the system and/or provide system control through feedback loops.

In certain embodiments, a method is further provided for real-time process monitoring and feedback during fractionation operations. This method includes continuously or intermittently measuring at least one of radiation exposure, fluid pressure, or flow rate within at least one channel of the microfluidic cassette using integrated sensors. Sensor data can be communicated to a control circuit configured to dynamically adjust the timing or sequence of valve actuation in response to detected variations in pressure or flow conditions. For example, if sensor readings indicate an unexpected increase in pressure within the microsphere conveying channel, the control circuit may alter the fractionation output valve actuation duration, alter pump operation, and/or initiate a temporary pause prior to advancing accumulated microspheres to a holding tank.

Furthermore, methods are included herein in which the application of positive and/or negative pressures is dynamically modulated over time according to a programmed cycle or in response to measured sensor data, allowing for tailored movement of microspheres and fluids during the different stages of dose fractionation and delivery as described herein elsewhere.

### Microspheres

Microspheres herein can include those with a combination of yttria, alumina, and silica. By way of example, in some embodiments, microspheres herein can include Y₂O₃-Al₂O₃-SiO₂ in a 40:20:40 wt. % ratio. It will be appreciated, however, that other types of microspheres are also contemplated herein including those with other materials.

In some embodiments, microspheres can be prepared by combining yttrium-89 with alumina and silica, in some cases also using a flame spheroidization method, and using neutron bombardment to convert Y-89 into the beta-emitting radioisotope Y-90. In various embodiments, the amount of beta radiation can exceed 400, 600, 800, 1000, 1500, 2000, 2500, 3000, 4000, 5000, 6000, 7000, 8000, 9000, 10000, or even 12000 Becquerel (Bq) per sphere or higher at the time of activity calibration (recognizing that the amount of radiation will drop after that point as the Y-90 radioisotope decays). In some embodiments, the microspheres can be provided in a master vial with activity of 3 Gigabecquerel (GBq) or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.35, 0.3, 0.25, 0.2, 0.15, 0.1, 0.05, or 0.01 GBq, or less at calibration time, or an amount falling within a range between any of the foregoing. It will be appreciated that the use of other isotopes is also contemplated herein.

It will be appreciated that fractionated dosages can vary based on factors including the type of tumor/tissue to be treated, location of the tumor/tissue to be treated, the number of fractionated doses to be provided, factors specific to a particular patient, and the like. In some embodiments the dosage of the therapy can be less than or equal to 5000 Gray (Gy), 4500 Gy, 4000 Gy, 3500 Gy, 3000 Gy, 2500 Gy, 2000 Gy, 1500 Gy, 1000 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 15 micrometers (μm) to about 35 μm. However, in some embodiments the microspheres can be somewhat smaller or larger.

The density of the microspheres can be quite high. In some embodiments, the density of the microspheres can be above 1.4, 1.8, 2, 2.5, or 3 grams/milliliter (g/mL), such as from 3.1 to 3.5 g/mL, or about 3.3 g/mL. By comparison, the density of water at room temperature is about 0.9978 g/mL. As such, the density of microspheres is much higher than an exemplary carrier fluid such as a saline solution which influences how readily such microspheres can settle out of a suspension.

It should be noted that, as used in this specification and the appended claims, the singular forms "a," "an," and "the" include plural referents unless the content clearly dictates otherwise. Thus, for example, reference to a composition containing "a compound" includes a mixture of two or more compounds. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase "configured" can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A fractionation system for microspheres for cancer therapy comprising:
a microfluidic cassette, the microfluidic cassette comprising
a microsphere conveying channel;
a microsphere dam, wherein the microsphere dam has a physical geometry configured to block microspheres from passing through the microsphere dam;
a weep channel, wherein the weep channel is separated from the microsphere conveying channel by the microsphere dam;
a fractionation output valve, wherein the fractionation output valve is in fluid communication with the microsphere conveying channel; and
a weep channel clearance valve, wherein the weep channel clearance valve is in fluid communication with the weep channel; and
a control circuit, wherein the control circuit is configured to control the fractionation output valve and the weep channel clearance valve.

2. The fractionation system for microspheres for cancer therapy of claim 1, wherein the microsphere dam has a height, width, and length dimension configured to allow fluid to pass into the weep channel while blocking passage of microspheres.

3. The fractionation system for microspheres for cancer therapy of claim 1, wherein the microsphere dam has a cross-sectional profile with a minimum aperture dimension size less than 20 micrometers.

4. The fractionation system for microspheres for cancer therapy of claim 1, further comprising a holding tank, wherein the holding tank is in fluid communication with the microsphere conveying channel as interrupted by the fractionation output valve.

5. The fractionation system for microspheres for cancer therapy of claim 4, further comprising:
a catheter connection port; and
a holding tank output valve, wherein the holding tank is in fluid communication with the catheter connection port as interrupted by the holding tank output valve.

6. The fractionation system for microspheres for cancer therapy of claim 1, the microfluidic cassette further comprising:
a fluidic layer; and
a capping layer, wherein the capping layer is disposed on the fluidic layer.

7. The fractionation system for microspheres for cancer therapy of claim 6, the capping layer comprising an embedded structural support.

8. The fractionation system for microspheres for cancer therapy of claim 1, further comprising:
a second microsphere dam, wherein the second microsphere dam has a physical geometry configured to block microspheres from passing through the microsphere dam; and
a second weep channel, wherein the second weep channel is separated from the microsphere conveying channel by the second microsphere dam.

9. The fractionation system for microspheres for cancer therapy of claim 1, the microsphere dam comprising a plurality of segments, wherein at least some of the plurality of segments are differently sized.

10. The fractionation system for microspheres for cancer therapy of claim 9, wherein segments of a smaller size from amongst the plurality of segments are oriented towards a proximal end of the microsphere conveying channel.

11. The fractionation system for microspheres for cancer therapy of claim 1, wherein the microsphere dam is oriented perpendicularly to the microsphere conveying channel.

12. The fractionation system for microspheres for cancer therapy of claim 1, wherein the microsphere dam is oriented non-perpendicularly to the microsphere conveying channel.

13. The fractionation system for microspheres for cancer therapy of claim 1, wherein the weep channel is deeper than the microsphere conveying channel.

14. The fractionation system for microspheres for cancer therapy of claim 1, wherein the weep channel is wider than the microsphere conveying channel.

15. The fractionation system for microspheres for cancer therapy of claim 1, wherein fluid in the weep channel flows in an opposite direction from the suspension in the microsphere conveying channel.

16. A fractionation system for microspheres for cancer therapy comprising:
a microfluidic cassette, the microfluidic cassette comprising
a fluidic layer, the fluidic layer defining a microsphere conveying channel;
a capping layer, the capping layer defining a weep channel;
a filter layer, the filter layer separating the weep channel from the microsphere conveying channel;
wherein the filter layer defines apertures configured to block microspheres from passing through the filter layer;
wherein the filter layer is disposed between the fluidic layer and the capping layer;
a fractionation output valve, wherein the fractionation output valve is in fluid communication with the microsphere conveying channel;
a weep channel clearance valve, wherein the weep channel clearance valve is in fluid communication with the weep channel; and
a control circuit, wherein the control circuit is configured to control the fractionation output valve and the weep channel clearance valve.

17. The fractionation system for microspheres for cancer therapy of claim 16, wherein the fractionation output valve is a pneumatically operated valve.

18. The fractionation system for microspheres for cancer therapy of claim 16, wherein the weep channel clearance valve is a pneumatically operated valve.

19. The fractionation system for microspheres for cancer therapy of claim 16, further comprising a holding tank, wherein the holding tank is in fluid communication with the microsphere conveying channel as interrupted by the fractionation output valve.

20. The fractionation system for microspheres for cancer therapy of claim 19, further comprising:
a catheter connection port; and
a holding tank output valve, wherein the holding tank is in fluid communication with the catheter connection port as interrupted by the holding tank output valve.
